(19) United States
(12) Patent Application Publication
(10) Pub. No.: US 2026/0108605 A1
(43) Pub. Date: Apr. 23, 2026

(54) ENGINEERED IMMUNE EFFECTOR CELL AND COMPOSITION AND USE THEREOF

(71) Applicant: SHANDONG SIMCERE BIOPHARMACEUTICAL CO., LTD., Yantai (CN)

(72) Inventors: Fuwei JIANG, Shanghai (CN); Tingting LIU, Shanghai (CN); Yifang WANG, Shanghai (CN); Cuiqing YANG, Shanghai (CN); Zhuoxiao CAO, Shanghai (CN)

(21) Appl. No.: 19/116,575

(22) PCT Filed: Sep. 27, 2023
(86) PCT No.: PCT/CN2023/121947

§ 371 Date: Mar. 28, 2025

(30) Foreign Application Priority Data
Sep. 29, 2022 (CN) 202211198709.8
Mar. 24, 2023 (CN) 202310311716.2

(51) Int. Cl. A61K 40/15; A61K 38/17; A61K 39/395; A61K 40/31; A61K 40/42; A61P 35/00; C07K 14/725; C07K 14/735; C07K 16/28

(52) U.S. Cl. CPC A61K 40/15; A61K 38/1774; A61K 39/39558; A61K 40/31; A61K 40/4211; A61P 35/00; C07K 14/7051; C07K 14/70535; C07K 16/2803; C07K 2319/00

(57) ABSTRACT

The present application relates to a genetically modified immune cell for expressing a recombinant human CD16 protein. The recombinant human CD16 protein has additions, deletions and replacements of, or any combination of the additions, deletions and replacements of one or more amino acids as compared to a wild-type amino acid sequence, and has enhanced shear resistance.

### RELATED APPLICATIONS

The present application claims priority to Chinese Patent Application No. 202211198709.8 filed on Sep. 29, 2022 and Chinese Patent Application No. 202310311716.2 filed on Mar. 24, 2023, which are incorporated herein by reference in their entirety for all purposes.

### TECHNICAL FIELD

The present application relates to the field of cells and particularly to engineered immune effector cells.

### BACKGROUND

CD16, also known as FcγRIII (low affinity immunoglobulin gamma Fc region receptor III), is a cell surface antigen (cluster of differentiation) on immune cells. It is a type III Fcγ receptor that can bind to the Fc fragment of immunoglobulin G (IgG). CD16 is divided into two proteins: CD16a (Fc region receptor III-A, FCGR3A) and CD16b (Fc region receptor III-B, FCGR3B). CD16a and CD16b share 96% sequence similarity in their extracellular antibody-binding domains. The amino acid sequence of CD16a is set forth in SEQ ID NO: 1, and its nucleotide sequence is set forth in SEQ ID NO: 2. CD16a is expressed on the surface of natural killer (NK) cells, mast cells, monocytes, macrophages, etc., while CD16b is only expressed on the surface of neutrophils. The amino acid sequence of CD16a is 254 amino acids in length (see SEQ ID NO: 1) and consists of a signal peptide, an extracellular domain with two Ig-like domains, a transmembrane region, and an intracellular domain.

As a member of the immunoglobulin superfamily (IgSF), CD16 is a low affinity IgG receptor that contains two extracellular Ig-like domains and is complementary to the receptor-binding region on the Fc of an antibody. Upon binding, it stimulates immune cells to initiate immune responses such as phagocytosis, antibody-dependent cell-mediated cytotoxicity (ADCC), and degranulation to attack target cells such as cancer cells or virus-infected cells. The binding of the CD16a on NK cells to an antibody stimulates the synthesis of CD25 and cytokines such as interferon-γ and tumor necrosis factor-α, thereby initiating ADCC. CD16a may also activate NK cells independently, without binding to an antibody, to lyse the target cell. Activation of NK cells by cytokines, target cell interactions, and/or tumor infiltration may result in CD16a cleavage and affect ADCC activity.

### SUMMARY

The present application prepares an immune cell expressing a recombinant CD16 protein. Compared to the prior art, the recombinant CD16 protein has an anti-cleavage ability and excellent binding activity to the Fc region of a human IgG1 antibody and can induce antibody-dependent cell-mediated cytotoxicity (ADCC).

In a first aspect, the present application provides a cell, wherein the cell is genetically modified to comprise or express an amino acid-modified CD16 protein. In some embodiments, the CD16 protein is derived from a human, a rat, a mouse, a monkey, a pig, a dog, or the like.

In some embodiments, a wild-type amino acid sequence of the CD16 protein may be selected from SEQ ID NOs: 1 and 3.

In some embodiments, the amino acid-modified CD16 protein is a CD16 protein comprising one or more amino acid mutations compared to a wild-type CD16 protein.

In some embodiments, the amino acid-modified CD16 protein comprises one or more amino acid additions, deletions, or substitutions or any combination of amino acid additions, deletions, and substitutions compared to the amino acid sequence of the wild-type CD16 protein.

In some embodiments, the one or more amino acid substitutions include a substitution of the glutamine residue at position 192 of SEQ ID NO: 3, a substitution of the leucine residue at position 194, a substitution of the valine residue at position 196, a substitution of the threonine residue at position 198, a substitution of the isoleucine residue at position 199, and/or a substitution of the serine residue at position 200.

In some embodiments, the one or more amino acid substitutions include Q192P, L194P, V196P, T198P, I199P, S200P, L194Y, L194V, L194K, L194I, A195V, V196E, V196D, V196K, V196N, V196G, V196R, V196Q, V196M, V196H, T191S, Q192N, Q192K, A195G, V196S, T198S, I199L, and/or S200T.

In some embodiments, the amino acid-modified CD16 protein comprises an amino acid sequence set forth in any one of SEQ ID NOs: 4-22 and 24-32 or an amino acid sequence having at least 80%, at least 85%, at least 90%, at least 95%, at least 96%, at least 97%, at least 98%, at least 99%, or 100% sequence identity to the amino acid sequence set forth in any one of SEQ ID NOs: 4-22 and 24-32.

In some embodiments, the cell further expresses a chimeric antigen receptor (CAR); preferably, the CAR specifically targets BCMA and GPRC5D; preferably, the CAR targeting BCMA and GPRC5D comprises an amino acid sequence set forth in SEQ ID NO: 46.

In some embodiments, the cell further expresses an IL-15 protein; preferably, the IL-15 protein comprises an amino acid sequence set forth in SEQ ID NO: 47.

In some embodiments, the cell expresses a fusion polypeptide comprising the CAR targeting BCMA and GPRC5D and the CD16 protein, wherein the fusion polypeptide comprises an amino acid sequence set forth in any one of SEQ ID NOs: 51-62 and 64-66 or an amino acid sequence having at least 80%, at least 85%, at least 90%, at least 95%, at least 96%, at least 97%, at least 98%, at least 99%, or 100% sequence identity to the amino acid sequence set forth in any one of SEQ ID NOs: 51-62 and 64-66.

In some embodiments, the cell is a T cell, a natural killer (NK) cell, a peripheral blood mononuclear cell (PBMC), a hematopoietic cell, a multipotent stem cell, or an embryonic stem cell.

In some embodiments, the cell is an NK cell. In a preferred embodiment, the cell is an NK92 cell.

In some embodiments, the amino acid-modified CD16 protein has an anti-cleavage ability.

In some embodiments, the amino acid-modified CD16 protein has ADAM17

In a second aspect, the present application provides a therapeutic composition, wherein the therapeutic composition comprises the cell according to any one of the above or a cell population comprising the cell.

In some embodiments, the therapeutic composition further comprises an iPSC cell population, an NK cell population, an NK92 cell population, or a T cell population.

In some embodiments, the iPSC cell population, the NK cell population, the NK92 cell population, or the T cell population is genetically modified to (i) specifically recognize a tumor antigen or (ii) specifically recognize a viral target.

In some embodiments, the therapeutic composition further comprises an additional therapeutic agent; preferably, the additional therapeutic agent is an antineoplastic agent; more preferably, the antineoplastic agent is a monoclonal antibody; more preferably, the monoclonal antibody is daratumumab or cetuximab.

In a third aspect, the present application provides a method, wherein the method comprises administering to a patient in need of such treatment a therapy comprising: administering to the patient the cell according to the first aspect or the therapeutic composition according to the second aspect. In some embodiments, the present application provides a method for treating a patient in need thereof, wherein the method comprises administering to the patient the cell according to the first aspect or the therapeutic composition according to the second aspect.

In a fourth aspect, the present application further provides use of the cell according to the first aspect or the therapeutic composition according to the second aspect for the preparation of a medicament for use in the method according to the third aspect.

In some embodiments, the method according to the third aspect or the medicament according to the fourth aspect is used for inhibiting tumor cell proliferation; in some embodiments, the tumor cell is a solid tumor cell or a hematologic tumor cell.

In some embodiments, the method according to the third aspect further comprises administering to the patient a therapeutic medicament; in some embodiments, the medicament may be selected from a monoclonal antibody, a polyclonal antibody, a small-molecule therapeutic agent, an antibody-drug conjugate, a cytokine, and the like; in some embodiments, the medicament inhibits tumor cell proliferation.

In some embodiments, the medicament according to the fourth aspect further comprises an additional therapeutic agent; preferably, the agent is selected from a monoclonal antibody, a polyclonal antibody, a small-molecule therapeutic agent, an antibody-drug conjugate, and a cytokine; more preferably, the agent inhibits tumor cell proliferation.

In a fifth aspect, the present application provides a medicament or kit comprising the cell according to the first aspect or the composition according to the second aspect.

In a sixth aspect, the present application provides an anti-cleavage recombinant CD16 protein, wherein the recombinant CD16 protein comprises one or more amino acid additions, deletions, or substitutions or any combination of amino acid additions, deletions, and substitutions compared to the amino acid sequence of a wild-type CD16 protein.

In some embodiments, the CD16 protein is derived from a human, a rat, a mouse, a monkey, a pig, or a dog.

In some embodiments, a wild-type amino acid sequence of the CD16 protein may be selected from SEQ ID NOs: 1 and 3.

In some embodiments, the one or more amino acid substitutions include a substitution of the glutamine residue at position 192 of SEQ ID NO: 3, a substitution of the leucine residue at position 194, a substitution of the valine residue at position 196, a substitution of the threonine residue at position 198, a substitution of the isoleucine residue at position 199, and/or a substitution of the serine residue at position 200.

In some embodiments, the one or more amino acid substitutions include Q192P, L194P, V196P, T198P, I199P, S200P, L194Y, L194V, L194K, L194I, A195V, V196E, V196D, V196K, V196N, V196G, V196R, V196Q, V196M, V196H, T191S, Q192N, Q192K, A195G, V196S, T198S, I199L, and/or S200T.

In some embodiments, the recombinant CD16 protein comprises an amino acid sequence set forth in any one of SEQ ID NOs: 4-22 and 24-32 or an amino acid sequence having at least 80%, at least 85%, at least 90%, at least 95%, at least 96%, at least 97%, at least 98%, at least 99%, or 100% sequence identity to the amino acid sequence set forth in any one of SEQ ID NOs: 4-22 and 24-32.

In a seventh aspect, the present application provides a fusion polypeptide, wherein the fusion polypeptide comprises any one of the recombinant CD16 proteins according to the sixth aspect.

In some embodiments, the fusion polypeptide further comprises a CAR sequence targeting BCMA and GPRC5D; preferably, the CAR sequence targeting BCMA and GPRC5D comprises an amino acid sequence set forth in SEQ ID NO: 46.

In some embodiments, the fusion polypeptide further comprises an IL-15 protein sequence; preferably, the IL-15 protein sequence comprises an amino acid sequence set forth in SEQ ID NO: 47.

In some embodiments, the fusion polypeptide comprises an amino acid sequence set forth in any one of SEQ ID NOs: 51-62 and 64-66 or an amino acid sequence having at least 80%, at least 85%, at least 90%, at least 95%, at least 96%, at least 97%, at least 98%, at least 99%, or 100% sequence identity to the amino acid sequence set forth in any one of SEQ ID NOs: 51-62 and 64-66.

In an eighth aspect, the present application provides a nucleic acid molecule, wherein the nucleic acid molecule encodes the recombinant CD16 protein according to the sixth aspect or the fusion polypeptide according to the seventh aspect.

In a ninth aspect, the present application provides an expression vector, wherein the expression vector comprises the nucleic acid molecule according to the eighth aspect.

In a tenth aspect, the present application provides a host cell, wherein the host cell comprises the expression vector according to the ninth aspect; preferably, the cell is a prokaryotic cell or a eukaryotic cell, such as a bacterium (E. coli), a fungus (yeast), an insect cell, or a mammalian cell (a CHO cell line or a 293T cell line).

### TERMINOLOGY AND DEFINITIONS

Unless otherwise defined herein, scientific and technical terms used in correlation with the present application shall have the meanings that are commonly understood by those skilled in the art.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “include”, “comprise”, and “have” herein are used interchangeably and are intended to indicate the inclusion of a solution, implying that there may be elements other than those listed in the solution. Meanwhile, it should be understood that the descriptions “include”, “comprise”, and “have” used herein also provide the solution of “consist of”. Illustratively, “a composition, comprising A and B” should be understood as the following technical solution: a composition consisting of A and B, and a composition containing other components in addition to A and B, all fall within the scope of the aforementioned “a composition”.

The term “and/or” used herein includes the meanings of “and”, “or”, and “all or any other combination of elements linked by the term”.

The term “genetically modified” herein has its plain and ordinary meaning and may include, but is not limited to, for example, a process of modifying an organism or a cell (e.g., a bacterium), a lymphocyte (e.g., a T cell or NK cell), a bacterial cell, a eukaryotic cell, an insect, a plant, or a mammal with genetic material (e.g., nucleic acid) that has been altered using genetic engineering techniques. For example, nucleic acid (e.g., DNA) can be inserted in the host genome by first isolating and copying the target genetic material using a molecular cloning method to generate a DNA sequence, or by synthesizing the DNA and subsequently inserting the construct into the host organism. Genes and gene expression can also be removed or “knocked out” using gene editing. Those skilled in the art can understand many techniques for knocking out a gene. Without limitation, a gene and/or gene expression can be knocked out using a technique such as RNA interference, CRISPRs, or TALENs. Gene targeting is a different technique that uses homologous recombination to alter an endogenous gene and can be used to delete a gene, remove an exon, add a gene, or introduce a point mutation. The term “genetically modified” as used herein also includes a cell or gene being engineered or naturally mutated to express a protein other than a wild-type protein.

Genetic modification performed by transduction is described herein. When read in light of the specification, “transduction” has its plain and ordinary meaning and may include, but is not limited to, for example, methods of transferring genetic material (e.g., DNA or RNA) to a cell through a vector. Commonly used techniques use viral vectors, electroporation, and chemical reagents to increase cell permeability. DNA can be transferred by a virus or a viral vector. As described herein, a method is provided for modifying immune cells (e.g., natural killer cells). Viral vectors may be derived from adenoviruses, adeno-associated viruses (AAV), retroviruses, and lentiviruses.

Various transduction techniques have been developed, and they utilize recombinant infectious virus particles for delivery. This represents a currently preferred cell transduction method. Viral vectors that can be used for transduction can include viral vectors derived from simian virus 40, adenoviruses, adeno-associated viruses (AAV), lentiviral vectors, and retroviruses. Thus, gene transfer and expression methods are numerous but essentially function to introduce and express genetic material in mammalian cells. Several of the above techniques can be used to transduce cells, including calcium phosphate transfection, protoplast fusion, electroporation, and infection with recombinant adenovirus, adeno-associated virus, lentiviral, or retroviral vectors. Lymphocytes have been successfully transduced by electroporation and retroviral or lentiviral infection. Thus, retroviral and lentiviral vectors can provide efficient methods for gene transfer in eukaryotic cells. Retroviral and lentiviral vectors provide efficient methods for gene transfer into lymphocytes (e.g., T cells and NK cells). In addition, retroviral or lentiviral integration takes place in a controlled manner and results in the stable integration of one or several copies of new genetic information per cell.

The term “amino acid-modified” as used herein includes one or more amino acid mutations, such as amino acid additions, deletions, or substitutions or any combination of the foregoing. The term “amino acid-modified protein” herein refers to a protein in which the amino acid sequence is altered, for example, one or more amino acid residues in the amino acid sequence are substituted, one or more amino acid residues are added, or one or more amino acid residues are deleted, etc.

The term “PMA” herein, short for phorbol-12-myristate-13-acetate or 12-O-tetradecanoylphorbol 13-acetate, is the most commonly used phorbol ester and can cause lysis or shedding of cell surface expressed proteins, including CD16a.

The term “CD16” herein is a low affinity Fc receptor found on the surface of an immune cell such as a natural killer cell, a neutrophil, or a monocyte, or a pluripotent stem cell or a differentiated cell generated from the pluripotent stem cell.

The term “natural killer cell” or “NK cell” herein has its plain and ordinary meaning and may include, but is not limited to, for example, natural killer cells from any tissue source and also includes natural killer cells produced using methods such as those described herein.

The term “hematopoietic cell” herein has its plain and ordinary meaning and may include, but is not limited to, for example, hematopoietic stem cells and hematopoietic progenitor cells.

The term “multipotent” herein has its plain and ordinary meaning and may include, but is not limited to, for example, when referring to a cell, meaning that the cell has the ability to differentiate into a cell of another cell type. In certain alternatives, a “multipotent stem cell” is a cell that has the ability to grow into a subset of cells of approximately 260 cell types of a mammalian body. Unlike a totipotent cell, a multipotent stem cell does not have the ability to form all cell types.

The term “therapeutic composition” herein refers to a formulation that exists in a form allowing the biological activity of the active ingredient contained therein to be effective and does not contain additional ingredients having unacceptable toxicity to a subject to which the pharmaceutical composition is administered.

The term “inhibits tumor cell proliferation” or “inhibiting tumor cell proliferation” herein has its plain and ordinary meaning and may include, but is not limited to, for example, slowing the growth of a tumor cell population, for example, by killing one or more of the tumor cells in the tumor cell population, for example, via contacting the cell or cell population described herein, or via contacting a population of tumor cells adjacent thereto with the cell or population of cells described herein.

### BRIEF DESCRIPTION OF THE DRAWINGS

FIG. 1: a schematic diagram of the structure of a plasmid expressing a recombinant CD16 protein.

FIG. 2A-FIG. 2A continued: anti-cleavage results for recombinant CD16 proteins with different point mutations. The positive proportions of CD16a on the surface of NK cells were determined by flow cytometry, and the changes in the expression levels of CD16a on the cell surface before and after PMA treatment were analyzed. The dark color indicates CD16a detection results before PMA treatment, and the light color indicates detection results after PMA treatment.

FIG. 2B: statistics for positive rates of CD16a on the cell surface before and after PMA treatment, wherein the dark color indicates CD16a detection results before PMA treatment, and the light color indicates detection results after PMA treatment.

FIG. 2C: the proportions of changes in positive rates of CD16a on the cell surface before and after PMA treatment.

FIG. 3: a schematic diagram of the structure of a plasmid of a human BCMA/GPRC5D-targeting CAR (A), and a schematic diagram of the structure of a human BCMA/GPRC5D-targeting CAR plasmid co-expressing a recombinant CD16a protein with a point mutation (B).

FIG. 4A: expression levels of a BCMA/GPRC5D CAR on the surface of human BCMA/GPRC5D-targeting CA-NK cells co-expressing recombinant CD16a proteins with point mutations, as determined by flow cytometry.

FIG. 4B: statistics for expression levels of a BCMA/GPRC5D CAR on the surface of human BCMA/GPRC5D-targeting CA-NK cells co-expressing recombinant CD16a proteins with point mutations.

FIG. 4C-FIG. 4C continued: anti-cleavage results for recombinant CD16 proteins with different point mutations. The positive proportions of CD16a on the surface of human BCMA/GPRC5D-targeting CA-NK cells co-expressing recombinant CD16a proteins with point mutations were determined by flow cytometry, and the changes in the expression levels of CD16a on the cell surface before and after PMA treatment were analyzed. The dark color indicates CD16a detection results after PMA treatment, and the light color indicates detection results before PMA treatment.

FIG. 5A: the ADCC activity of human BCMA/GPRC5D-targeting CAR-NK cells co-expressing recombinant CD16a proteins with point mutations after 4 h of co-culture at E:T=1:1.

FIG. 5B: the ADCC activity of human BCMA/GPRC5D-targeting CAR-NK cells co-expressing recombinant CD16a proteins with point mutations after 4 h of co-culture at E:T=5:1.

FIG. 5C: the ADCC activity of human BCMA/GPRC5D-targeting CAR-NK cells co-expressing recombinant CD16a proteins with point mutations after 24 h of co-culture at E:T=1:1.

FIG. 6A: expression levels of recombinant CD16a proteins with point mutations on the surface of NK92 cells, as determined by flow cytometry.

FIG. 6B: the ADCC activity of NK92 cells expressing recombinant CD16a proteins with point mutations after 24 h of co-culture at E:T=1:1.

FIG. 7: a schematic diagram of the structure of a fusion polypeptide of a human CD19-targeting CAR and a recombinant CD16a protein with a point mutation.

FIG. 8: the ADCC activity of human CD19-targeting CAR-NK cells expressing a recombinant CD16a protein with a point mutation prepared by iPSC induction and editing after 4 h of co-culture at different E:T ratios.

### DETAILED DESCRIPTION

The present application will be further described with reference to specific examples, and the advantages and features of the present application will become more apparent with the description. Experimental procedures without specified conditions in the examples are conducted according to conventional conditions or conditions recommended by the manufacturers. Reagents or instruments without specified manufacturers used herein are conventional products that are commercially available.

The examples of the present application are exemplary only, and do not limit the scope of the present application in any way. It will be understood by those skilled in the art that various modifications or substitutions may be made to the technical solutions of the present application in form and details without departing from the spirit and scope of the present application, and that these modifications and substitutions shall fall within the protection scope of the present application.

Unless otherwise stated, the target cells MOLP8 (Nanjing Cobioer, Cat #CBP60562) and HCT-116 (Nanjing Cobioer, Cat #CBP60028) described in the following examples were transfected with the luciferase gene to express luciferase. The luciferase reporter gene assay reagent was used for determining the fluorescence intensity and reflecting the cell viability and the killing effect of NK cells.

### Example 1. Expression Vector Construction

1.1. Introduction of Site-Directed Mutations into CD16a Protein

The amino acid sequence of the CD16a protein (NP_000560.7, NCBI database):

(SEQ ID NO: 1)
MWQLLLPTALLLLVSAGMRTEDLPKAVVFLEPQWYRVLEKDSVTLKCQG
AYSPEDNSTQWFHNESLISSQASSYFIDAATVDDSGEYRCQTNLSTLSD
PVQLEVHIGWLLLQAPRWVFKEEDPIHLRCHSWKNTALHKVTYLQNGKG
RKYFHHNSDFYIPKATLKDSGSYFCRGLFGSKNVSSETVNITITQGLAV
STISSFFPPGYQVSFCLVMVLLFAVDTGLYESV
KTNIRSSTRDWKDHKFKWRKDPQDK.

Note: The signal peptide is single-underlined, the transmembrane region is double-underlined, the intracellular domain is italicized, and the rest is the extracellular domain with two Ig-like domains. The nucleotide sequence encoding the CD16a protein (NM_000569.8, NCBI database):

(SEQ ID NO: 2)
ATGTGGCAGCTGCTCCTCCCAACTGCTCTGCTACTTCTAGTTTCAGCTG
GCATGCGGACTGAAGATCTCCCAAAGGCTGTGGTGTTCCTGGAGCCTCA
ATGGTACAGGGTGCTCGAGAAGGACAGTGTGACTCTGAAGTGCCAGGGA
GCCTACTCCCCTGAGGACAATTCCACACAGTGGTTTCACAATGAGAGCC
TCATCTCAAGCCAGGCCTCGAGCTACTTCATTGACGCTGCCACAGTCGA
CGACAGTGGAGAGTACAGGTGCCAGACAAACCTCTCCACCCTCAGTGAC
CCGGTGCAGCTAGAAGTCCATATCGGCTGGCTGTTGCTCCAGGCCCCTC
GGTGGGTGTTCAAGGAGGAAGACCCTATTCACCTGAGGTGTCACAGCTG
GAAGAACACTGCTCTGCATAAGGTCACATATTTACAGAATGGCAAAGGC
AGGAAGTATTTTCATCATAATTCTGACTTCTACATTCCAAAAGCCACAC
TCAAAGACAGCGGCTCCTACTTCTGCAGGGGGCTTTTTGGGAGTAAAAA
TGTGTCTTCAGAGACTGTGAACATCACCATCACTCAAGGTTTGGCAGTG
TCAACCATCTCATCATTCTTTCCACCTGGGTACCAAGTCTCTTTCTGCT
TGGTGATGGTACTCCTTTTTGCAGTGGACACAGGACTATATTTCTCTGT
GAAGACAAACATTCGAAGCTCAACAAGAGACTGGAAGGACCATAAATTT
AAATGGAGAAAGGACCCTCAAGACAAATGA.

The mutations introduced were as follows: Q192P, L194P, V196P, T198P, I199P, S200P, L194Y, L194V, L194K, L194I, A195V, V196E, V196D, V196K, V196N, V196G, V196R, V196Q, V196M, V196H, T191S, Q192N, Q192K, A195G, V196S, T198S, I199L, and S200T. The mutated sequences are shown in Table 1.

TABLE 1
Recombinant CD16a amino acid sequences with a point mutation

| SEQ | Insertion | | | |
|---|---|---|---|---|
| ID | sequence | Mutation | Plasmid/ | |
| NO | No. | site | cell No. | Amino acid sequence |
| 3 | 16 | With | 1600 | MWQLLLPTALLLLVSAGMRTEDLPKAVVFL |
| | | natural | | EPQWYRVLEKDSVTLKCQGAYSPEDNSTQW |
| | | mutation | | FHNESLISSQASSYFIDAATVDDSGEYRCQTN |
| | | F176V | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAVSTISSFFPPGYQVSFCLVMVLL |
| | | | | FAVDTGLYFSVKTNIRSSTRDWKDHKFKWR |
| | | | | KDPQDK |
| 4 | 16-2 | Q192P | 1602 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITPGLAVSTISSFFPPGYQVSFCLVMVLL |
| | | | | FAVDTGLYFSVKTNIRSSTRDWKDHKFKWR |
| | | | | KDPQDK |
| 5 | 16-4 | L194P | 1604 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGPAVSTISSFFPPGYQVSFCLVMVLL |
| | | | | FAVDTGLYFSVKTNIRSSTRDWKDHKFKWR |
| | | | | KDPQDK |
| 6 | 16-6 | V196P | 1606 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAPSTISSFFPPGYQVSFCLVMVLL |
| | | | | FAVDTGLYFSVKTNIRSSTRDWKDHKFKWR |
| | | | | KDPQDK |
| 7 | 16-7 | T198P | 1607 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAVSPISSFFPPGYQVSFCLVMVLL |
| | | | | FAVDTGLYFSVKTNIRSSTRDWKDHKFKWR |
| | | | | KDPQDK |
| 8 | 16-8 | I199P | 1608 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAVSTPSSFFPPGYQVSFCLVMVL |
| | | | | LFAVDTGLYFSVKTNIRSSTRDWKDHKFKW |
| | | | | RKDPQDK |
| 9 | 16-9 | S200P | 1609 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAVSTIPSFFPPGYQVSFCLVMVLL |
| | | | | FAVDTGLYFSVKTNIRSSTRDWKDHKFKWR |
| | | | | KDPQDK |
| 10 | 16-10 | L194Y | 1610 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGYAVSTISSFFPPGYQVSFCLVMVLL |
| | | | | FAVDTGLYFSVKTNIRSSTRDWKDHKFKWR |
| | | | | KDPQDK |
| 11 | 16-11 | L194V | 1611 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGVAVSTISSFFPPGYQVSFCLVMVLL |
| | | | | FAVDTGLYFSVKTNIRSSTRDWKDHKFKWR |
| | | | | KDPQDK |
| 12 | 16-12 | L194K | 1612 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGKAVSTISSFFPPGYQVSFCLVMVL |
| | | | | LFAVDTGLYFSVKTNIRSSTRDWKDHKFKW |
| | | | | RKDPQDK |
| 13 | 16-15 | A195V | 1615 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLVVSTISSFFPPGYQVSFCLVMVLL |
| | | | | FAVDTGLYFSVKTNIRSSTRDWKDHKFKWR |
| | | | | KDPQDK |
| 14 | 16-19 | V196E | 1619 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAESTISSFFPPGYQVSFCLVMVLL |
| | | | | FAVDTGLYFSVKTNIRSSTRDWKDHKFKWR |
| | | | | KDPQDK |
| 15 | 16-20 | V196D | 1620 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLADSTISSFFPPGYQVSFCLVMVL |
| | | | | LFAVDTGLYFSVKTNIRSSTRDWKDHKFKW |
| | | | | RKDPQDK |
| 16 | 16-21 | V196K | 1621 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAKSTISSFFPPGYQVSFCLVMVL |
| | | | | LFAVDTGLYFSVKTNIRSSTRDWKDHKFKW |
| | | | | RKDPQDK |
| 17 | 16-22 | V196N | 1622 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLANSTISSFFPPGYQVSFCLVMVL |
| | | | | LFAVDTGLYFSVKTNIRSSTRDWKDHKFKW |
| | | | | RKDPQDK |
| 18 | 16-23 | V196G | 1623 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAGSTISSFFPPGYQVSFCLVMVL |
| | | | | LFAVDTGLYFSVKTNIRSSTRDWKDHKFKW |
| | | | | RKDPQDK |
| 19 | 16-24 | V196R | 1624 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLARSTISSFFPPGYQVSFCLVMVLL |
| | | | | FAVDTGLYFSVKTNIRSSTRDWKDHKFKWR |
| | | | | KDPQDK |
| 20 | 16-25 | V196Q | 1625 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAQSTISSFFPPGYQVSFCLVMVL |
| | | | | LFAVDTGLYFSVKTNIRSSTRDWKDHKFKW |
| | | | | RKDPQDK |
| 21 | 16-26 | V196M | 1626 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAMSTISSFFPPGYQVSFCLVMVL |
| | | | | LFAVDTGLYFSVKTNIRSSTRDWKDHKFKW |
| | | | | RKDPQDK |
| 22 | 16-27 | V196H | 1627 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAHSTISSFFPPGYQVSFCLVMVL |
| | | | | LFAVDTGLYFSVKTNIRSSTRDWKDHKFKW |
| | | | | RKDPQDK |
| 23 | 16-30 | S197P | 1630 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAVPTISSFFPPGYQVSFCLVMVLL |
| | | | | FAVDTGLYFSVKTNIRSSTRDWKDHKFKWR |
| | | | | KDPQDK |
| 24 | 16-31 | T191S | 1631 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITISQGLAVSTISSFFPPGYQVSFCLVMVLL |
| | | | | FAVDTGLYFSVKTNIRSSTRDWKDHKFKWR |
| | | | | KDPQDK |
| 25 | 16-32 | Q192N | 1632 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITNGLAVSTISSFFPPGYQVSFCLVMVLL |
| | | | | FAVDTGLYFSVKTNIRSSTRDWKDHKFKWR |
| | | | | KDPQDK |
| 26 | 16-33 | Q192K | 1633 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITKGLAVSTISSFFPPGYQVSFCLVMVLL |
| | | | | FAVDTGLYFSVKTNIRSSTRDWKDHKFKWR |
| | | | | KDPQDK |
| 27 | 16-35 | L194I | 1635 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGIAVSTISSFFPPGYQVSFCLVMVLL |
| | | | | FAVDTGLYFSVKTNIRSSTRDWKDHKFKWR |
| | | | | KDPQDK |
| 28 | 16-36 | A195G | 1636 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLGVSTISSFFPPGYQVSFCLVMVL |
| | | | | LFAVDTGLYFSVKTNIRSSTRDWKDHKFKW |
| | | | | RKDPQDK |
| 29 | 16-37 | V196S | 1637 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLASSTISSFFPPGYQVSFCLVMVLL |
| | | | | FAVDTGLYFSVKTNIRSSTRDWKDHKFKWR |
| | | | | KDPQDK |
| 30 | 16-38 | T198S | 1638 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAVSSISSFFPPGYQVSFCLVMVLL |
| | | | | FAVDTGLYFSVKTNIRSSTRDWKDHKFKWR |
| | | | | KDPQDK |
| 31 | 16-39 | I199L | 1639 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAVSTLSSFFPPGYQVSFCLVMVL |
| | | | | LFAVDTGLYFSVKTNIRSSTRDWKDHKFKW |
| | | | | RKDPQDK |
| 32 | 16-40 | S200T | 1640 | MWQLLLPTALLLLVSAGMRTEDLPKAVVFL |
| | | | | EPQWYRVLEKDSVTLKCQGAYSPEDNSTQW |
| | | | | FHNESLISSQASSYFIDAATVDDSGEYRCQTN |
| | | | | LSTLSDPVQLEVHIGWLLLQAPRWVFKEEDP |
| | | | | IHLRCHSWKNTALHKVTYLQNGKGRKYFHH |
| | | | | NSDFYIPKATLKDSGSYFCRGLVGSKNVSSET |
| | | | | VNITITQGLAVSTITSFFPPGYQVSFCLVMVLL |
| | | | | FAVDTGLYFSVKTNIRSSTRDWKDHKFKWR |
| | | | | KDPQDK |

### 1.2. Plasmid Construction

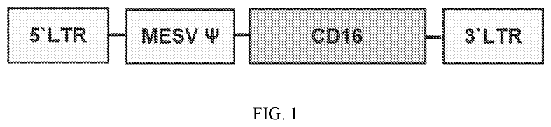

In this example, CD16a protein-expressing retroviral shuttle plasmids 1600, 1602, 1604, 1606, 1607, 1608, 1609, 1610, 1611, 1612, 1615, 1619, 1620, 1621, 1622, 1623, 1624, 1625, 1626, 1627, 1629, 1630, 1631, 1632, 1633, 1635, 1636, 1637, 1638, 1639, and 1640 were constructed using a retroviral vector template and conventional molecular biology methods in the art according to the schematic diagram of a plasmid shown in FIG. 1.

First, insertion sequences 16, 16-2, 16-4, 16-6, 16-7, 16-8, 16-9, 16-10, 16-11, 16-12, 16-15, 16-19, 16-20, 16-21, 16-22, 16-23, 16-24, 16-25, 16-26, 16-27, 16-30, 16-31, 16-32, 16-33, 16-35, 16-36, 16-37, 16-38, 16-39, and 16-40 were separately synthesized, and EcoRI and SalI enzyme cutting sites, as well as corresponding vector homologous sequences, were added to both ends, respectively. The insertion sequence of the control plasmid 1600 was a recombinant CD16a protein with the natural point mutation F176V. The control plasmid 1629 was a retroviral shuttle plasmid without an insertion sequence. The insertion sequence 16-30 of the positive control plasmid 1630 was from the patent US 2020/0017570 A1.

The retroviral vector template was digested using the restriction endonucleases EcoRI (Thermo, Cat #FD0274) and SalI (Thermo, Cat #FD0644), and the linear plasmids were recovered and purified through agarose gel electrophoresis. The polynucleotide sequences synthesized in the above steps were each ligated to a linearized vector through recombinase 5× In-FusionHD enzyme (TaKaRa, Cat #ST0344). The reaction system was as follows: 2 μL of synthesized polynucleotide fragment (50 ng/μL), 1 μL of linearized plasmid (50 ng/μL), 2 μL of 5×HD In Fusion enzyme, and 5 μL of ddH₂O. The reaction system was gently pipetted to ensure thorough mixing, centrifuged briefly, and left to react at 50° C. for 15 min. 10 μL of recombinant reaction product was added to 100 μL of bacterial competent cells, and the mixture was left on ice for 5 min. The transformed bacterium solution was uniformly applied onto LB plates containing 50 μg/mL kanamycin, and the plates were incubated in an inverted position in a thermostatic incubator for 12-16 h. From each plate, 3-5 clones were randomly picked for sequencing identification. The bacterium solution confirmed to be correct by sequencing was transferred to 100 mL of an LB liquid culture medium containing 50 μg/mL kanamycin, and the mixture was incubated overnight at 37° C. Plasmid extraction was performed using an MN endotoxin-free plasmid extraction Midi kit (MN, Cat #740420.50). After quantification, the extracted plasmids were diluted to 1000 ng/μL with endotoxin-free ultrapure water to obtain the CD16a protein-expressing retroviral shuttle plasmids described above.

### 1.3. Retrovirus Preparation

293T cells (Cell Bank of Type Culture Collection of Chinese Academy of Sciences, Cat #GNHu17) were inoculated onto a 100-mm culture dish and cultured using a DMEM culture medium (Gibco, Cat #10566016) containing 10% FBS (Gibco, Cat #10099141). When 293T cells covered approximately 70% of the dish surface, plasmid transfection was performed: A CD16a protein-expressing retroviral shuttle plasmid was mixed with a packaging plasmid, and the mixture was then added to 1.2 mL of Opti-MEM culture medium (Thermofisher Scientific, Cat #31985070). Then 35 μL of Fugene HD (Promega, Cat #04709691001) was added and well mixed, and the resulting mixture was incubated at room temperature for 15 min and added to a 293T cell culture medium in good condition. The resulting mixture was then incubated in a thermostatic incubator (37° C., 5% CO₂). After 48 h, the 293T cell supernatant was collected, filtered through a 0.45 μm filter membrane, and concentrated for later use.

### Example 2. PMA-Induced CD16a Cleaving Effect Assay

### 2.1. NK Cell Culture and Viral Infection

Fresh PBMCs were centrifuged at room temperature at 500 g for 7 min. After the culture supernatant was discarded, NK cells were isolated using the method provided by the Human NK Cell isolation kit (Stemcell, Cat #17955). The isolated NK cells were activated using K562 cells as follows: On day 0, counting was performed using the AO/PI staining method; cells were mixed in an NK-to-K562 ratio of 1:2 and added to a Non-Treated 6-well plate (the culture medium was an NK cell culture medium (Miltenyi Biotec, Cat #130-114-429) containing 200 IU/mL human IL2) at 2 mL/well, and the plate was incubated in an incubator (37° C., 5% CO₂). On day 4, 3 mL of culture medium was added to each well. On day 5, a 24-well plate was coated overnight at 4° C. with a 7 μg/mL RetroNectin reagent (Takara, Cat #T202), with 500 μL per well. On day 6, corresponding retroviruses were added to the 24-well plate. The 24-well plate with the viruses was centrifuged at 2000 g at 4-8° C. for 60 min. The virus supernatant was discarded. NK cells were counted and added to the 24-well plate at 3E5 cells/well, and the plate was centrifuged at 400 g at room temperature for 5 min. The 24-well plate was incubated in an incubator (37° C., 5% CO₂). On day 7, the transfected NK cells were transferred into a Non-Treated 6-well plate for further culture.

### 2.2. CD16a Shedding Assay

On day 5 after viral infection, the expression levels of cell surface CD16a were determined. Before the flow cytometry assays were started, 100 ng/mL PMA (STEMCELL, Cat #74044) was added to the culture medium. After 40 min, 3 centrifugal washes were performed using flow cytometry staining buffer (Gibco, Cat #00-4222-26). After 1 h of incubation at 4° C. with 2 μg/mL CD16a nanobody (from the patent PCT/CN2022/101713, VHH-Fc 12-05, prepared in-house), 3 centrifugal washes were performed using flow cytometry staining buffer. After 1 h of incubation at 4° C. with the secondary antibody Alexa Fluor 647 AffiniPure Goat Anti-Human IgG Fcγ fragment specific antibody (Jackson ImmunoReearch, Cat #109-605-098), diluted in 1:800, 3 centrifugal washes were performed using flow cytometry staining buffer. The cells were suspended in 100 μL of FACS buffer and assayed on a flow cytometer (BD, CANTOII), and the results were analyzed.

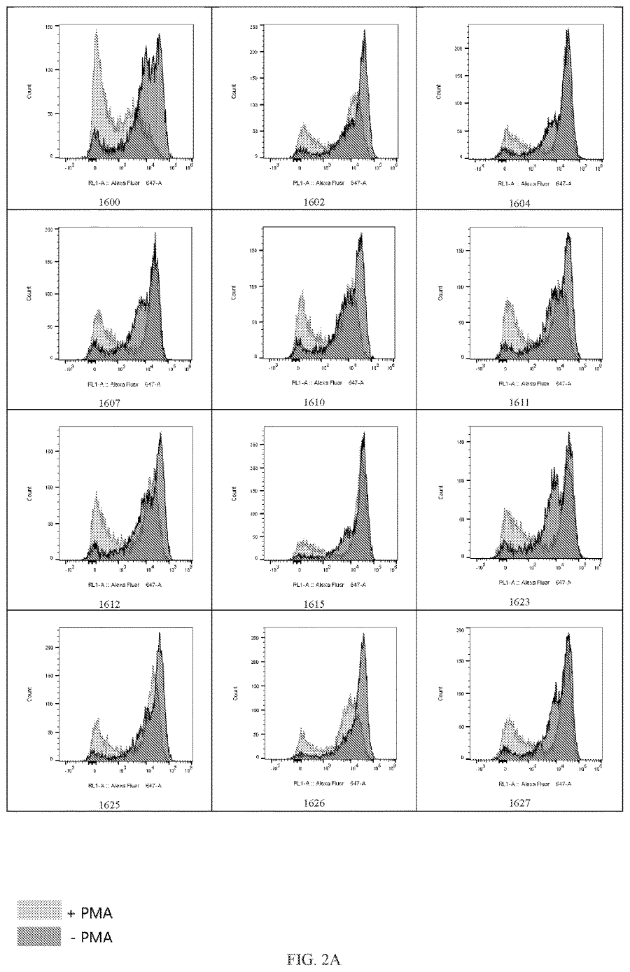

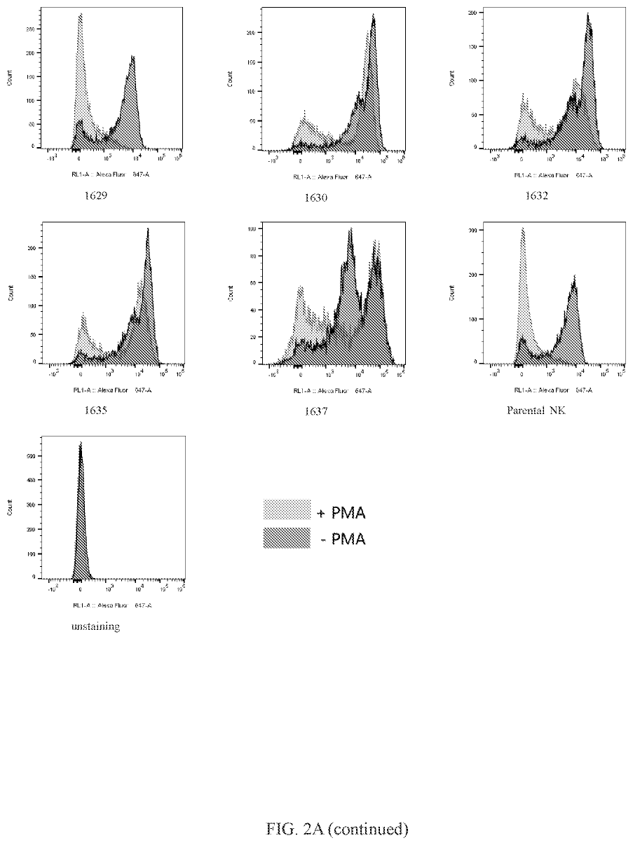

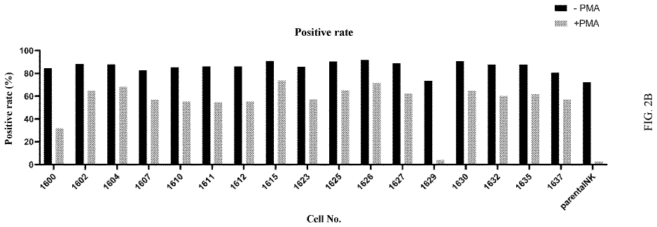

The results are shown in FIGS. 2A and 2B. After PMA addition, CD16a was nearly completely shed from the surface of primary NK cells (Parental NK) and 1629 cells which were infected with an empty virus, while the positive rates of CD16a proteins with point mutations on the cell surface significantly increased. As shown in Table 2, the positive rates of CD16a on the surface of cells 1602, 1604, 1607, 1610, 1611, 1612, 1615, 1623, 1625, 1626, 1627, 1632, 1635, and 1637 were 64.7%, 68.4%, 56.9%, 55.4%, 54.9%, 55.3%, 73.8%, 57.3%, 65.1%, 71.6%, 62.2%, 60.1%, 61.9%, and 57.1%, respectively, which were close to or higher than the positive rate of the positive control 1630 (64.9%). Unstaining indicates flow cytometry assay results for primary NK cells without primary antibody addition.

TABLE 2
Expression levels of cell surface CD16a
before and after PMA treatment

| | Before PMA treatment | After PMA treatment |
|---|---|---|
| Cell No. | (CD16a positive %) | (CD16a positive %) |
| 1600 | 84.5 | 32.0 |
| 1602 | 88.3 | 64.7 |
| 1604 | 88.0 | 68.4 |
| 1607 | 82.7 | 56.9 |
| 1610 | 85.4 | 55.4 |
| 1611 | 86.1 | 54.9 |
| 1612 | 86.2 | 55.3 |
| 1615 | 91.0 | 73.8 |
| 1623 | 85.7 | 57.3 |
| 1625 | 90.2 | 65.1 |
| 1626 | 91.6 | 71.6 |
| 1627 | 88.9 | 62.2 |
| 1629 | 73.4 | 3.95 |
| 1630 | 90.9 | 64.9 |
| 1632 | 87.9 | 60.1 |
| 1635 | 87.8 | 61.9 |
| 1637 | 80.8 | 57.1 |
| Parental NK | 72.3 | 2.72 |

As shown in FIG. 2C, after PMA addition, the changes in the positive rates of CD16a proteins with point mutations on the cell surface significantly decreased. As shown in Table 3, the changes in the positive rates of CD16a on the surface of cells 1602, 1604, 1607, 1610, 1611, 1612, 1615, 1623, 1625, 1626, 1627, 1632, 1635, and 1637 were 26.7%, 22.3%, 31.2%, 35.1%, 36.2%, 35.8%, 18.9%, 33.1%, 27.8%, 21.8%, 30.0%, 31.6%, 29.5%, and 29.3%, respectively, which were close to or lower than the change in the positive rate of the positive control 1630 (28.6%). Unstaining indicates flow cytometry assay results for primary NK cells without primary antibody addition.

TABLE 3
Changes in the positive rates of cell surface
CD16a before and after PMA treatment

| | After PMA treatment (change in |
|---|---|
| Cell No. | positive rate of surface CD16a %) |
| 1600 | 62.1 |
| 1602 | 26.7 |
| 1604 | 22.3 |
| 1607 | 31.2 |
| 1610 | 35.1 |
| 1611 | 36.2 |
| 1612 | 35.8 |
| 1615 | 18.9 |
| 1623 | 33.1 |
| 1625 | 27.8 |
| 1626 | 21.8 |
| 1627 | 30.0 |
| 1629 | 94.6 |
| 1630 | 28.6 |
| 1632 | 31.6 |
| 1635 | 29.5 |
| 1637 | 29.3 |
| Parental NK | 96.2 |

Thus, it was demonstrated that introducing point mutation Q192P, L194P, T198P, L194Y, L194V, L194K, A195V, V196G, V196Q, V196M, V196H, Q192N, L194I, or V196S into CD16a effectively blocked cleavage-induced CD16a shedding.

### Example 3. Expression and Purification of Recombinant Fragments of Extracellular Domains of CD16a Proteins with Point Mutations

Recombinant pCDNA3.4 plasmids were constructed according to the amino acid sequences shown in Table 4. The proteins were expressed using the Expi293™ expression system and purified. The purification results are shown in Table 5.

TABLE 4
The amino acid sequences of recombinant fragments of the extracellular
domains of CD16a proteins with point mutations

| SEQ | Protein | Point | |
|---|---|---|---|
| ID NO | No. | mutation | Amino acid sequence |
| 33 | 1600WE | / | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLFGSKNVSS |
| | | | ETVNITITQGLAVSTISSFFPPGYQHHHHHH |
| 34 | 1600E | F176V | MWQLLLPTALLLLVSAGMRTEDLPKAVVFL |
| | | (Natural | EPQWYRVLEKDSVTLKCQGAYSPEDNSTQW |
| | | mutation) | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGLAVSTISSFFPPGYQHHHHHH |
| 35 | 1602E | Q192P | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITPGLAVSTISSFFPPGYQHHHHHH |
| 36 | 1604E | L194P | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGPAVSTISSFFPPGYQHHHHHH |
| 37 | 1615E | A195V | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGLVVSTISSFFPPGYQHHHHHH |
| 38 | 1623E | V196G | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGLAGSTISSFFPPGYQHHHHHH |
| 39 | 1625E | V196Q | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGLAQSTISSFFPPGYQHHHHHH |
| 40 | 1626E | V196M | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGLAMSTISSFFPPGYQHHHHHH |
| 41 | 1627E | V196H | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGLAHSTISSFFPPGYQHHHHHH |
| 42 | 1630E | S197P | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGLAVPTISSFFPPGYQHHHHHH |
| 43 | 1632E | Q192N | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITNGLAVSTISSFFPPGYQHHHHHH |
| 44 | 1635E | L194I | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGIAVSTISSFFPPGYQHHHHHH |
| 45 | 1637E | V196S | MWQLLLPTALLLLVSAGMRTEDLPKAVVFL |
| | | | EPQWYRVLEKDSVTLKCQGAYSPEDNSTQW |
| | | | FHNESLISSQASSYFIDAATVDDSGEYRCQTN |
| | | | LSTLSDPVQLEVHIGWLLLQAPRWVFKEEDP |
| | | | IHLRCHSWKNTALHKVTYLQNGKGRKYFH |
| | | | HNSDFYIPKATLKDSGSYFCRGLVGSKNVSS |
| | | | ETVNITITQGLASSTISSFFPPGYQHHHHHH |

As shown in Table 5, the purified recombinant proteins all exhibited purities greater than 90% as determined by SEC analysis. Compared to the wild-type CD16a protein 1600WE and 1630E, which contained point mutation S197P, most recombinant proteins with other point mutations exhibited significantly improved purities. The purity of protein 1604E was 99.76%, and the purities of proteins 1615E, 1623E, 1625E, and 1626E were 100%. This indicates that separately introducing L194P, A195V, V196G, V196Q, and V196M can significantly improve the purity of the extracellular domain fragment of the CD16a protein.

TABLE 5
Purity results for recombinant fragments of the extracellular
domains of CD16a proteins with point mutations

| Protein No. | SEC purity (%) |
|---|---|
| 1600WE | 90.46 |
| 1600E | 94.52 |
| 1602E | 94.50 |
| 1604E | 99.76 |
| 1615E | 100.00 |
| 1623E | 100.00 |
| 1625E | 100.00 |
| 1626E | 100.00 |
| 1627E | 95.47 |
| 1630E | 94.74 |
| 1632E | 92.48 |
| 1635E | 95.97 |
| 1637E | 95.49 |

### Example 4. Determination of Affinities of Recombinant CD16a Proteins with Point Mutations for IgG1

The final concentration of the antibody anti-FITC IgG1 (prepared in-house) was adjusted to 2 μg/mL using HBS-EP+ buffer (Cytiva, Cat #BR-1006-69), and the antibody was added to a 96-well plate (Greiner Bio-one, Cat #210100581) at 600 μL/well. Anti-FITC IgG1 was captured using a ProteinA chip (Cytiva, Cat #29-1275-56) on a Biacore 8K system (Cytiva, Cat #Biacore 8K). The purified CD16a proteins in Example 3 were serially diluted from 6000 nM to 187.5 nM using HBS-EP+ buffer and added to the 96-well plate (Greiner Bio-one, Cat #210100581) at 600 μL/well, and their affinities were determined by following the instructions of Biacore 8K Control Software. The results are shown in Table 6.

TABLE 6
The affinities of CD16a proteins with
different point mutations for IgG1

| | Binding kinetics | | |
|---|---|---|---|
| Protein | ka (1/Ms) | kd (1/s) | KD (M) |
| 1600WE | 9.95E+04 | 2.58E−01 | 2.60E−06 |
| 1600E | 2.45E+05 | 1.19E−01 | 4.84E−07 |
| 1602E | 2.14E+05 | 1.19E−01 | 5.57E−07 |
| 1604E | 2.36E+05 | 1.13E−01 | 4.77E−07 |
| 1615E | 1.96E+05 | 1.13E−01 | 5.76E−07 |
| 1623E | 2.51E+05 | 1.05E−01 | 4.18E−07 |
| 1625E | 2.70E+05 | 1.12E−01 | 4.15E−07 |
| 1626E | 2.40E+05 | 1.14E−01 | 4.76E−07 |
| 1627E | 2.67E+05 | 1.07E−01 | 4.02E−07 |
| 1630E | 2.60E+05 | 1.17E−01 | 4.48E−07 |
| 1632E | 2.44E+05 | 1.17E−01 | 4.79E−07 |
| 1635E | 2.40E+05 | 1.21E−01 | 5.06E−07 |
| 1637E | 2.57E+05 | 1.01E−01 | 3.91E−07 |

As shown in Table 6, the naturally occurring point mutation F176V (4.84E-07) enhanced the affinity of CD16a for IgG1 Fc compared to wild-type CD16a (with a KD of 2.60E-06); compared to point mutation F176V, the additionally introduced point mutations showed no effect on affinity.

Thus, it was demonstrated that introducing point mutations Q192P, L194P, A195V, V196G, V196Q, V196M, V196H, Q192N, L194I, or V196S into CD16a not only enabled it to have an anti-cleavage ability but also effectively ensured its affinity for the Fc of the IgG1 antibody.

### Example 5. Assays for ADCC Activity of Human BCMA/GPRC5D-Targeting CAR-NK Cells Co-Expressing Recombinant CD16a Proteins with Point Mutations

### 5.1. Expression Vector Construction

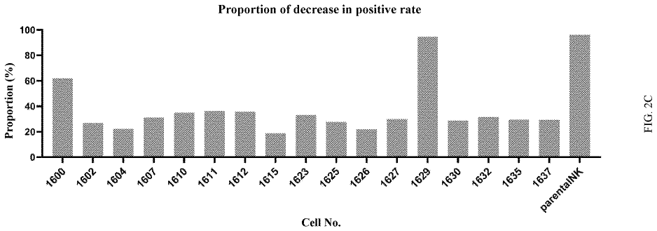

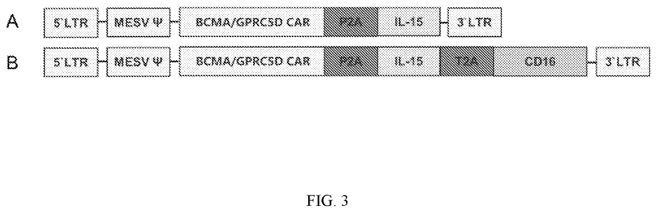

A retroviral shuttle plasmid BCAR was constructed as a negative control using the method in Example 1 according to the schematic diagram of a plasmid shown in A of FIG. 3. The BCAR contained a CAR targeting human BCMA and GPRC5D (BCMA/GPRC5D CAR, SEQ ID NO: 46) and human IL-15 (SEQ ID NO: 47). Meanwhile, according to the schematic diagram of a plasmid shown in B of FIG. 3, human CD16a sequences 16, 16-2, 16-4, 16-7, 116-10, 16-11, 16-12, 16-15, 16-23, 16-25, 16-26, 16-27, 16-30, 16-32, 16-33, 16-35, and 16-37 were introduced into the BCAR to construct retroviral shuttle plasmids B00, B02, B04, B07, B10, B11, B12, B15, B23, B25, B26, B27, B30, B32, B33, B35, and B37. B30 was used as a positive control. The related amino acid sequences are shown in Tables 7 and 8.

TABLE 7
The amino acid sequences of the BCAR and its elements

| SEQ | | |
|---|---|---|
| ID NO | Name | Amino acid sequence |
| 46 | BCMA/GPRC5D | MALPVTALLLPLALLLHAARPDIQMTQSPSSLSASVGD |
| | CAR | RVTITCKASQDVRTAVAWYQQKPGKSPKLLIYWASTRH |
| | | TGVPDRFSGSGSGTDFTLTISSLQPEDVATYYCQQHYST |
| | | PLTFGQGTKLEIKGGGGSGGGGSGGGGSQVQLKESGP |
| | | VLVKPTETLTLTCTVSGFSLTSYGVNWIRQPPGKALEW |
| | | LAVIWSGGNTDYNAAFRSRLTISKDNSKSQVVLTMTN |
| | | MDPVDTATYYCARGQLGRPYWYFDVWGQGTTVTVSS |
| | | GGGGSGGGGSGGGGSGGGGSEVQLVESGGGLVQPGG |
| | | SLRLSCAASESISSIHIMAWYRQAPGKQRELVAGIRNDG |
| | | STVYVDSVKGRFTISRDNAKNSVYLQMNSLRAEDTAV |
| | | YYCNADQGFGSYSEWERRSRWGQGTTVTVSSTTTPAP |
| | | RPPTPAPTIASQPLSLRPEACRPAAGGAVHTRGLDFACD |
| | | SPFFFCCFIAVAMGIRFIIMVAIWSAVFLNSWRRKRKEK |
| | | QSETSPKEFLTIYEDVKDLKTRRNHEQEQTFPGGGSTIY |
| | | SMIQSQSSAPTSQEPAYTLYSLIQPSRKSGSRKRNHSPSF |
| | | NSTIYEVIGKSQPKAQNPARLSRKELENFDVYSRVKFSR |
| | | SADAPAYQQGQNQLYNELNLGRREEYDVLDKRRGRDP |
| | | EMGGKPRRKNPQEGLYNELQKDKMAEAYSEIGMKGE |
| | | RRRGKGHDGLYQGLSTATKDTYDALHMQALPPR |
| 47 | Human IL-15 | MRISKPHLRSISIQCYLCLLLNSHFLTEAGIHVFILGCFS |
| | | AGLPKTEANWVNVISDLKKIEDLIQSMHIDATLYTESD |
| | | VHPSCKVTAMKCFLLELQVISLESGDASIHDTVENLIIL |
| | | ANNSLSSNGNVTESGCKECEELEEKNIKEFLQSFVHIVQ |
| | | MFINTS |
| 48 | P2A | ATNFSLLKQAGDVEENPGP |
| 49 | T2A | EGRGSLLTCGDVEENPGP |
| 50 | BCMA/GPRC5D | MALPVTALLLPLALLLHAARPDIQMTQSPSSLSASVGD |
| | CAR and | RVTITCKASQDVRTAVAWYQQKPGKSPKLLIYWASTRH |
| | human IL-15 | TGVPDRFSGSGSGTDFTLTISSLQPEDVATYYCQQHYST |
| | (BCAR) | PLTFGQGTKLEIKGGGGSGGGGSGGGGSQVQLKESGP |
| | | VLVKPTETLTLTCTVSGFSLTSYGVNWIRQPPGKALEW |
| | | LAVIWSGGNTDYNAAFRSRLTISKDNSKSQVVLTMTN |
| | | MDPVDTATYYCARGQLGRPYWYFDVWGQGTTVTVSS |
| | | GGGGSGGGGSGGGGSGGGGSEVQLVESGGGLVQPGG |
| | | SLRLSCAASESISSIHIMAWYRQAPGKQRELVAGIRNDG |
| | | STVYVDSVKGRFTISRDNAKNSVYLQMNSLRAEDTAV |
| | | YYCNADQGFGSYSEWERRSRWGQGTTVTVSSTTTPAP |
| | | RPPTPAPTIASQPLSLRPEACRPAAGGAVHTRGLDFACD |
| | | SPFFFCCFIAVAMGIRFIIMVAIWSAVFLNSWRRKRKEK |
| | | QSETSPKEFLTIYEDVKDLKTRRNHEQEQTFPGGGSTIY |
| | | SMIQSQSSAPTSQEPAYTLYSLIQPSRKSGSRKRNHSPSF |
| | | NSTIYEVIGKSQPKAQNPARLSRKELENFDVYSRVKFSR |
| | | SADAPAYQQGQNQLYNELNLGRREEYDVLDKRRGRDP |
| | | EMGGKPRRKNPQEGLYNELQKDKMAEAYSEIGMKGE |
| | | RRRGKGHDGLYQGLSTATKDTYDALHMQALPPRGSG |
| | | ATNFSLLKQAGDVEENPGPMRISKPHLRSISIQCYLCLL |
| | | LNSHFLTEAGIHVFILGCFSAGLPKTEANWVNVISDLKK |
| | | IEDLIQSMHIDATLYTESDVHPSCKVTAMKCFLLELQVI |
| | | SLESGDASIHDTVENLIILANNSLSSNGNVTESGCKECE |
| | | ELEEKNIKEFLQSFVHIVQMFINTS |

TABLE 8
The amino acid sequences of fusion polypeptides of a human BCMA/GPRC5D-targeting
CAR and human CD16 proteins with point mutations

| | CD16a | | | |
|---|---|---|---|---|
| SEQ ID | insertion | Mutation | Plasmid/NK | |
| NO | sequence No. | site | cell No. | Amino acid sequence |
| 50 | / | / | BCAR | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTS |
| 51 | 16 | With | B00 | MALPVTALLLPLALLLHAARPDIQMTQSPSSLS |
| | | natural | | ASVGDRVTITCKASQDVRTAVAWYQQKPGKSPK |
| | | mutation | | LLIYWASTRHTGVPDRFSGSGSGTDFTLTISSLQP |
| | | F176V | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGLAVSTISSFFPPGYQVSF |
| | | | | CLVMVLLFAVDTGLYFSVKTNIRSSTRDWKDHK |
| | | | | FKWRKDPQDK |
| 52 | 16-2 | Q192P | B02 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITPGLAVSTISSFFPPGYQVSF |
| | | | | CLVMVLLFAVDTGLYFSVKTNIRSSTRDWKDHK |
| | | | | FKWRKDPQDK |
| 53 | 16-4 | L194P | B04 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGPAVSTISSFFPPGYQVSF |
| | | | | CLVMVLLFAVDTGLYFSVKTNIRSSTRDWKDHK |
| | | | | FKWRKDPQDK |
| 54 | 16-7 | T198P | B07 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGLAVSPISSFFPPGYQVSF |
| | | | | CLVMVLLFAVDTGLYFSVKTNIRSSTRDWKDHK |
| | | | | FKWRKDPQDK |
| 55 | 16-10 | L194Y | B10 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGYAVSTISSFFPPGYQVSF |
| | | | | CLVMVLLFAVDTGLYFSVKTNIRSSTRDWKDHK |
| | | | | FKWRKDPQDK |
| 56 | 16-11 | L194V | B11 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGVAVSTISSFFPPGYQVSF |
| | | | | CLVMVLLFAVDTGLYFSVKTNIRSSTRDWKDHK |
| | | | | FKWRKDPQDK |
| 57 | 16-12 | L194K | B12 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGKAVSTISSFFPPGYQVSF |
| | | | | CLVMVLLFAVDTGLYFSVKTNIRSSTRDWKDHK |
| | | | | FKWRKDPQDK |
| 58 | 16-15 | A195V | B15 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGLVVSTISSFFPPGYQVSF |
| | | | | CLVMVLLFAVDTGLYFSVKTNIRSSTRDWKDHK |
| | | | | FKWRKDPQDK |
| 59 | 16-23 | V196G | B23 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGLAGSTISSFFPPGYQVSF |
| | | | | CLVMVLLFAVDTGLYFSVKTNIRSSTRDWKDHK |
| | | | | FKWRKDPQDK |
| 60 | 16-25 | V196Q | B25 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGLAQSTISSFFPPGYQVSF |
| | | | | CLVMVLLFAVDTGLYFSVKTNIRSSTRDWKDHK |
| | | | | FKWRKDPQDK |
| 61 | 16-26 | V196M | B26 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGLAMSTISSFFPPGYQVSF |
| | | | | CLVMVLLFAVDTGLYFSVKTNIRSSTRDWKDHK |
| | | | | FKWRKDPQDK |
| 62 | 16-27 | V196H | B27 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGLAHSTISSFFPPGYQVSF |
| | | | | CLVMVLLFAVDTGLYFSVKTNIRSSTRDWKDHK |
| | | | | FKWRKDPQDK |
| 63 | 16-30 | S197P | B30 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGLAVPTISSFFPPGYQVSF |
| | | | | CLVMVLLFAVDTGLYFSVKTNIRSSTRDWKDHK |
| | | | | FKWRKDPQDK |
| 64 | 16-32 | Q192N | B32 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITNGLAVSTISSFFPPGYQVSF |
| | | | | CLVMVLLFAVDTGLYFSVKTNIRSSTRDWKDHK |
| | | | | FKWRKDPQDK |
| 65 | 16-35 | L194I | B35 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGIAVSTISSFFPPGYQVSFC |
| | | | | LVMVLLFAVDTGLYFSVKTNIRSSTRDWKDHKF |
| | | | | KWRKDPQDK |
| 66 | 16-37 | V196S | B37 | MALPVTALLLPLALLLHAARPDIQMTQSPSSLS |
| | | | | ASVGDRVTITCKASQDVRTAVAWYQQKPGKSPK |
| | | | | LLIYWASTRHTGVPDRFSGSGSGTDFTLTISSLQP |
| | | | | EDVATYYCQQHYSTPLTFGQGTKLEIKGGGGSG |
| | | | | GGGSGGGGSQVQLKESGPVLVKPTETLTLTCTV |
| | | | | SGFSLTSYGVNWIRQPPGKALEWLAVIWSGGNT |
| | | | | DYNAAFRSRLTISKDNSKSQVVLTMTNMDPVD |
| | | | | TATYYCARGQLGRPYWYFDVWGQGTTVTVSS |
| | | | | GGGGSGGGGSGGGGSGGGGSEVQLVESGGGLV |
| | | | | QPGGSLRLSCAASESISSIHIMAWYRQAPGKQRE |
| | | | | LVAGIRNDGSTVYVDSVKGRFTISRDNAKNSVY |
| | | | | LQMNSLRAEDTAVYYCNADQGFGSYSEWERRS |
| | | | | RWGQGTTVTVSSTTTPAPRPPTPAPTIASQPLSLR |
| | | | | PEACRPAAGGAVHTRGLDFACDSPFFFCCFIAVA |
| | | | | MGIRFIIMVAIWSAVFLNSWRRKRKEKQSETSPK |
| | | | | EFLTIYEDVKDLKTRRNHEQEQTFPGGGSTIYSM |
| | | | | IQSQSSAPTSQEPAYTLYSLIQPSRKSGSRKRNHS |
| | | | | PSFNSTIYEVIGKSQPKAQNPARLSRKELENFDV |
| | | | | YSRVKFSRSADAPAYQQGQNQLYNELNLGRREE |
| | | | | YDVLDKRRGRDPEMGGKPRRKNPQEGLYNELQ |
| | | | | KDKMAEAYSEIGMKGERRRGKGHDGLYQGLST |
| | | | | ATKDTYDALHMQALPPRGSGATNFSLLKQAGD |
| | | | | VEENPGPMRISKPHLRSISIQCYLCLLLNSHFLTE |
| | | | | AGIHVFILGCFSAGLPKTEANWVNVISDLKKIED |
| | | | | LIQSMHIDATLYTESDVHPSCKVTAMKCFLLELQ |
| | | | | VISLESGDASIHDTVENLIILANNSLSSNGNVTES |
| | | | | GCKECEELEEKNIKEFLQSFVHIVQMFINTSLEG |
| | | | | GGEGRGSLLTCGDVEENPGPRMWQLLLPTALLL |
| | | | | LVSAGMRTEDLPKAVVFLEPQWYRVLEKDSVT |
| | | | | LKCQGAYSPEDNSTQWFHNESLISSQASSYFIDA |
| | | | | ATVDDSGEYRCQTNLSTLSDPVQLEVHIGWLLL |
| | | | | QAPRWVFKEEDPIHLRCHSWKNTALHKVTYLQ |
| | | | | NGKGRKYFHHNSDFYIPKATLKDSGSYFCRGLV |
| | | | | GSKNVSSETVNITITQGLASSTISSFFPPGYQVSF |
| | | | | CLVMVLLFAVDTGLYFSVKTNIRSSTRDWKDHK |
| | | | | FKWRKDPQDK |

### 5.2. CD16a Shedding Assay

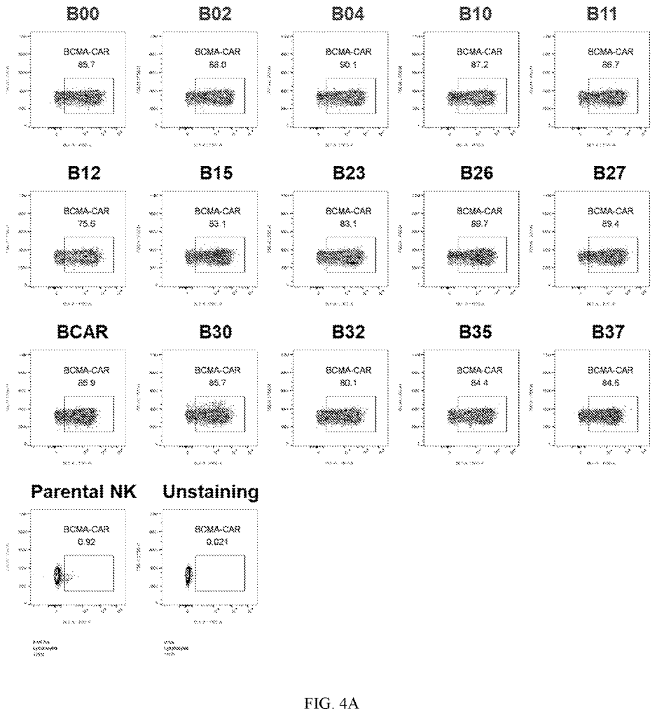

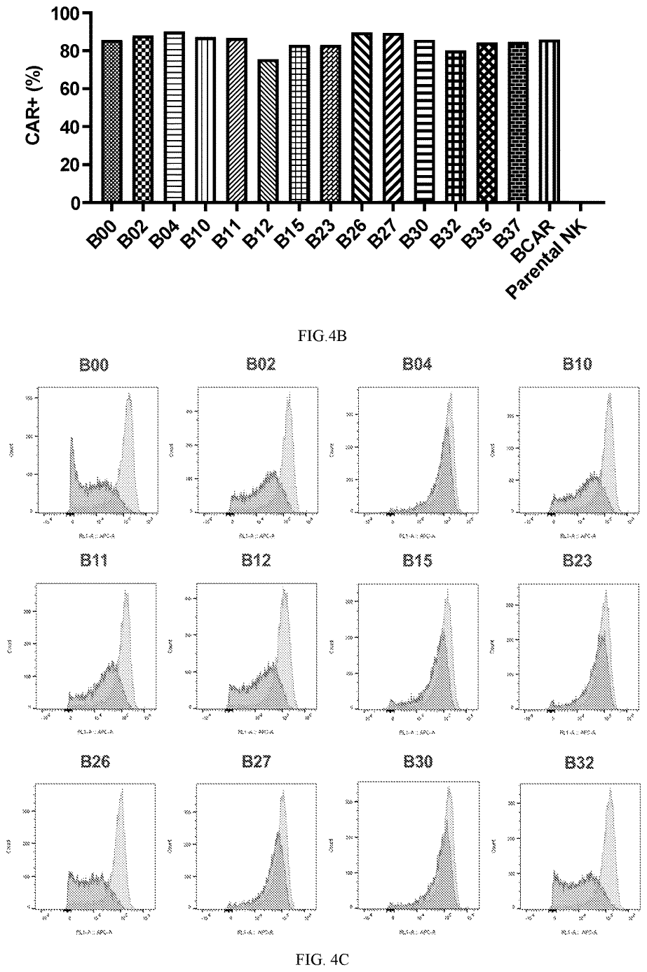

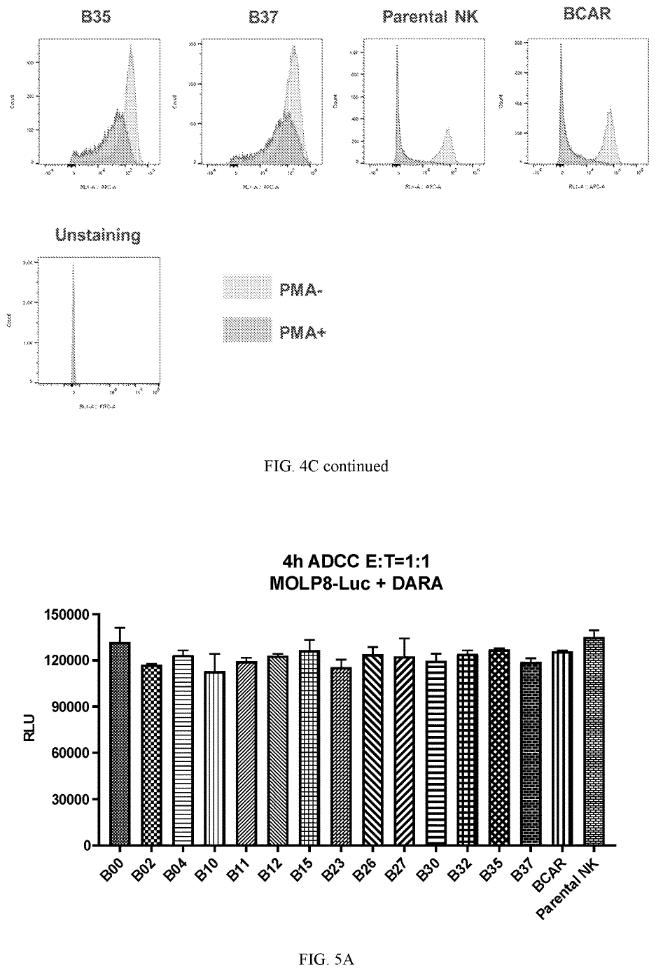

Cell culture, viral infection, PMA stimulation, and protein expression assays were performed using the methods in Example 2. On day 5 post-infection, the expression levels of the BCMA/GPRC5D CAR were determined. The expression levels of the BCMA CAR on the NK cells were determined through flow cytometry assays using FITC-labeled BCMA protein (Acrobiosystems, Cat #BCA-HF254), and the results are shown in FIGS. 4A and 4B. The expression levels of the BCMA/GPRC5D CAR on the surface of the CAR-NK cells were similar.

On day 6 after viral infection, the expression levels of CD16a on the surface of the NK cells were determined. Before the CD16a shedding assays were started, 100 ng/ml PMA (STEMCELL, Cat #74044) was added to the culture medium. After 60 min, 3 centrifugal washes were performed using flow cytometry staining buffer (Gibco, Cat #00-4222-26). After 1 h of incubation at 4° C. with 2 μg/mL CD16a nanobody VHH-Fc 12-05 (from the patent PCT/CN2022/101713, VHH-Fc 12-05, prepared in-house), 3 centrifugal washes were performed using flow cytometry staining buffer. After 1 h of incubation at 4° C. with the secondary antibody Alexa Fluor 647 AffiniPure Goat Anti-Human IgG Fcγ fragment specific (Jackson ImmunoReearch, Cat #109-605-098), diluted in 1:800, 3 centrifugal washes were performed using flow cytometry staining buffer. The cells were suspended in 100 μL of FACS buffer and assayed on a flow cytometer (BD, CANTOII), and the results were analyzed.

The results are shown in FIG. 4C. After PMA addition, CD16a was nearly completely shed from the surface of primary NK cells (Parental NK) and cells only expressing the BCMA/GPRC5D CAR, while the positive rates of CD16a proteins with point mutations on the cell surface significantly increased. The positive rates of CD16a on the surface of cells B04, B15, B23, and B27 did not significantly change and were close to that of the positive control B30. Unstaining indicates flow cytometry assay results for primary NK cells without primary antibody addition.

### 5.3. ADCC Activity Assays

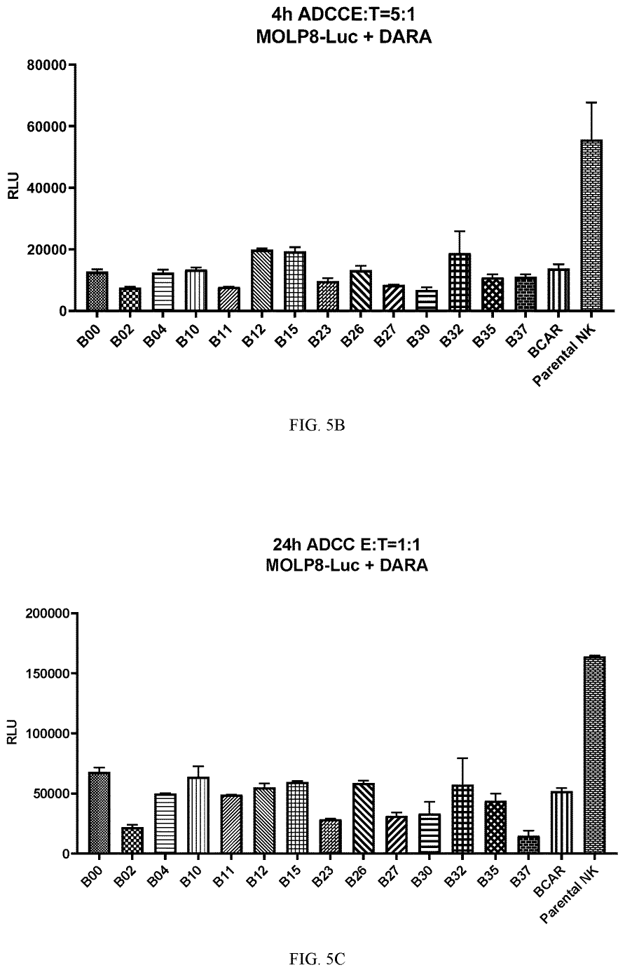

The ADCC activity of human BCMA/GPRC5D-targeting CAR-NK cells co-expressing CD16a proteins with point mutations was verified using an experiment of the CAR-NK cells killing MOLP8-Luc cells in combination with daratumumab (Biointron, Cat #B625101). The target MOLP8-Luc cells were incubated with 10 nM daratumumab at 4° C. for 30 min. The mixture was centrifuged, and the supernatant was discarded. The MOLP8-Luc cells were resuspended in RPMI 1640 culture medium (Gibco, Cat #11875093) at a density of 2×10⁴ cells/100 μL for later use. The effector-to-target ratio (E:T) was set to 1:1 or 5:1 for the experiment. 100 μL of CAR-NK cells and 100 μL of MOLP8-Luc cells were added to an opaque 96-well plate, and replicate wells were set. The cells were co-cultured in a cell incubator for 4 h or 24 h. After the co-culture was completed, 50 μL of the fluorescein bioluminescent substrate D-luciferin (Yeasen Biotechnology, Cat #115144-35-9) was added, and the plate was incubated at room temperature in a dark place for 10 min. Then numerical values were read using a microplate reader (PE, Ensight-HH3400) and analyzed, and the results are shown in FIG. 5. The lower the numerical value, the smaller the number of remaining target cells, and the stronger the ADCC activity. As shown in FIG. 5A, after being co-cultured at E:T=1:1 for 4 h, the CAR-NK cells exhibited similar ADCC activity to one another: there was no significant difference in the number of remaining target cells. As shown in FIG. 5B, after being co-cultured at E:T=5:1 for 4 h, the CAR-NK cells differed in ADCC activity: B02, B11, B23, and B27 exhibited stronger ADCC activity, and their numbers of remaining cells were close to that of the positive control B30. As shown in FIG. 5C, after being co-cultured at E:T=1:1 for 24 h, the CAR-NK cells differed in ADCC activity: B02, B23, B27, and B37 exhibited stronger ADCC activity, and their numbers of remaining cells were significantly smaller than that of the positive control B30.

### Example 6. Assays for ADCC Activity of NK92 Cells Expressing Recombinant CD16a Proteins with Point Mutations

### 6.1. NK Cell Culture and Viral Infection

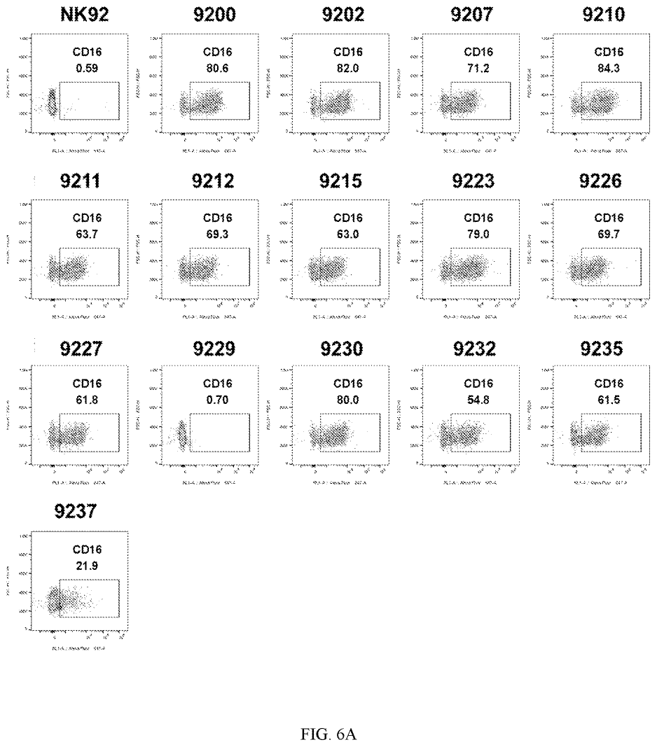

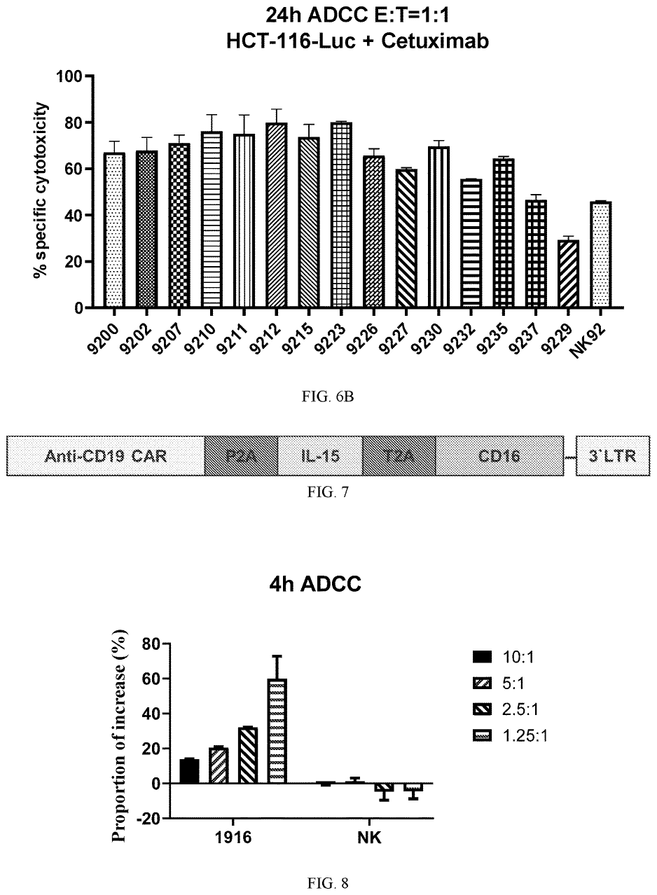

NK92 cells were infected with the retroviruses containing plasmids 1600, 1602, 1607, 1610, 1611, 1612, 1615, 1623, 1626, 1627, 1629, 1630, 1632, 1635, and 1637 in Example 1 using the method of Example 1 to prepare the following NK92 cells expressing recombinant CD16a proteins with point mutations: 9200, 9202, 9207, 9210, 9211, 9212, 9215, 9223, 9226, 9227, 9229, 9230, 9232, 9235, and 9237. Five days after infection, the expression levels of CD16a were determined, and the results are shown in FIG. 6A. All proteins were expressed on the surface of NK92 cells.

TABLE 9
The numbering of NK92 cells expressing recombinant CD16a proteins
with point mutations and the corresponding mutation sites

| Plasmid No. | Mutation site | NK92 cell No. |
|---|---|---|
| 1600 | Natural mutation F176V | 9200 |
| 1602 | Q192P | 9202 |
| 1607 | T198P | 9207 |
| 1610 | L194Y | 9210 |
| 1611 | L194V | 9211 |
| 1612 | L194K | 9212 |
| 1615 | A195V | 9215 |
| 1623 | V196G | 9223 |
| 1626 | V196M | 9226 |
| 1627 | V196H | 9227 |
| 1629 | Without insertion sequences | 9229 |
| 1630 | S197P | 9230 |
| 1632 | Q192N | 9232 |
| 1635 | L194I | 9235 |
| 1637 | V196S | 9237 |

### 6.2. ADCC Activity Assays

The ADCC activity of NK92 cells expressing CD16a proteins with point mutations was verified using an experiment of the NK92 cells killing HCT-116-Luc cells in combination with cetuximab (Biointron, Cat #B139201). HCT116-Luc cells were resuspended in RPMI 1640 culture medium (Gibco, Cat #11875093) at a density of 2×10⁴ cells/100 μL for later use. The effector-to-target ratio (E:T) was set to 1:1 for the experiment. 100 μL of NK92 cells and 100 μL of HCT-116-Luc cells were added to an opaque 96-well plate, and replicate wells were set. The cells were co-cultured in a cell incubator for 24 h. After the co-culture was completed, 50 μL of the fluorescein bioluminescent substrate D-luciferin (Yeasen Biotechnology, Cat #115144-35-9) was added, and the plate was incubated at room temperature in a dark place for 10 min. Then numerical values were read using a microplate reader (PE, Ensight-HH3400) and analyzed, and the results are shown in FIG. 6B. After being co-cultured at E:T=1:1 for 24 h, the cell groups differed significantly in ADCC activity: 9207, 9210, 9211, 9212, 9215, and 9223 exhibited stronger ADCC activity than the positive control 9230, and their proportions of killed cells were higher. The mutated CD16a proteins exhibited excellent ADCC-stimulating effects on NK92 cells.

### Example 7. Assay for ADCC Activity of Human CD19-Targeting CAR-NK Cells Co-Expressing Recombinant CD16a Protein Prepared by iPSC Induction and Editing

### 7.1. CAR-NK Cell Preparation

iPSC-derived NK cells were prepared using a disclosed induction method of induced pluripotent stem cells (iPSCs), such as Zhu, H., Kaufman, D. S. (2019). An Improved Method to Produce Clinical-Scale Natural Killer Cells from Human Pluripotent Stem Cells. In: Kaneko, S. (eds) In Vitro Differentiation of T-Cells. Methods in Molecular Biology, vol 2048. The NK cells obtained by induction were further edited using a gene editing method of CAR NK cells, such as Burnight E R, et. al. CRISPR-Cas9-Mediated Correction of the 1.02 kb Common Deletion in CLN3 in Induced Pluripotent Stem Cells from Patients with Batten Disease. CRISPR J. 2018 February; 1(1): 75-87, to prepare NK cells co-expressing a recombinant CD16a protein (with point mutation V196G) and a human CD19 CAR (1916 cells for short). The inserted gene contained nucleotides encoding a fusion polypeptide of a human CD19-targeting CAR, human IL-15, and a recombinant human CD16a protein, and its structure is schematically shown in FIG. 7. The related amino acid sequences are shown in Table 10. The amino acid sequence of the recombinant human CD16a protein with point mutation V196G is set forth in SEQ ID NO: 18.

TABLE 10
The amino acid sequences of proteins related to a fusion polypeptide of a human
CD19-targeting CAR and a human CD16 protein with a point mutation

| SEQ ID NO | Name | Amino acid sequence |
|---|---|---|
| 67 | CD19 CAR | MALPVTALLLPLALLLHAARPLEEIVLTQSPATLSLSPGERA |
| | | TLSCSASSSISSNYLHWYQQKPGQAPRFLIYRTSNLASGIPA |
| | | RFSGSGSGTDFTLTISSLEPEDFAVYYCQQASSIPRMFTFGQ |
| | | GTKLEIKGSTSGSGKPGSGEGSTKGEVQLQQSGAEVKKPG |
| | | ASVKVSCKASGYTFTNYWMHWVRQRPGQGLEWMGEIDP |
| | | SDNYANYNQEFQGRVTITVDKSASTAYMELSSLRSEDTAV |
| | | YYCARHDGYFGALDYWGQGTTVTVSSGSFVPVFLPAKRT |
| | | TTPAPRPPTPAPTIASQPLSLRPEACRPAAGGAVHTRGLDFA |
| | | CDIYIWAPLAGTCGVLLLSLVITLYCRSKRSRLLHSDYMNM |
| | | TPRRPGPTRKHYQPYAPPRDFAAYRSRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQG |
| | | LSTATKDTYDALHMQALPPR |
| 68 | Human IL-15 | MRISKPHLRSISIQCYLCLLLNSHFLTEAGIHVFILGCFSAGL |
| | | PKTEANWVNVISDLKKIEDLIQSMHIDATLYTESDVHPSCK |
| | | VTAMKCFLLELQVISLESGDASIHDTVENLIILANNSLSSNG |
| | | NVTESGCKECEELEEKNIKEFLQSFVHIVQMFINTS |
| 69 | P2A | ATNFSLLKQAGDVEENPGP |
| 70 | T2A | EGRGSLLTCGDVEENPGP |
| 71 | CD19 CAR | MALPVTALLLPLALLLHAARPLEEIVLTQSPATLSLSPGERA |
| | and human | TLSCSASSSISSNYLHWYQQKPGQAPRFLIYRTSNLASGIPA |
| | IL-15 | RFSGSGSGTDFTLTISSLEPEDFAVYYCQQASSIPRMFTFGQ |
| | | GTKLEIKGSTSGSGKPGSGEGSTKGEVQLQQSGAEVKKPG |
| | | ASVKVSCKASGYTFTNYWMHWVRQRPGQGLEWMGEIDP |
| | | SDNYANYNQEFQGRVTITVDKSASTAYMELSSLRSEDTAV |
| | | YYCARHDGYFGALDYWGQGTTVTVSSGSFVPVFLPAKRT |
| | | TTPAPRPPTPAPTIASQPLSLRPEACRPAAGGAVHTRGLDFA |
| | | CDIYIWAPLAGTCGVLLLSLVITLYCRSKRSRLLHSDYMNM |
| | | TPRRPGPTRKHYQPYAPPRDFAAYRSRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQG |
| | | LSTATKDTYDALHMQALPPRGSGATNFSLLKQAGDVEENP |
| | | GPMRISKPHLRSISIQCYLCLLLNSHFLTEAGIHVFILGCFSA |
| | | GLPKTEANWVNVISDLKKIEDLIQSMHIDATLYTESDVHPS |
| | | CKVTAMKCFLLELQVISLESGDASIHDTVENLIILANNSLSS |
| | | NGNVTESGCKECEELEEKNIKEFLQSFVHIVQMFINTS |
| 72 | CD19 CAR, | MALPVTALLLPLALLLHAARPLEEIVLTQSPATLSLSPGERA |
| | human IL- | TLSCSASSSISSNYLHWYQQKPGQAPRFLIYRTSNLASGIPA |
| | 15, and | RFSGSGSGTDFTLTISSLEPEDFAVYYCQQASSIPRMFTFGQ |
| | CD16 with | GTKLEIKGSTSGSGKPGSGEGSTKGEVQLQQSGAEVKKPG |
| | point | ASVKVSCKASGYTFTNYWMHWVRQRPGQGLEWMGEIDP |
| | mutation | SDNYANYNQEFQGRVTITVDKSASTAYMELSSLRSEDTAV |
| | V196G | YYCARHDGYFGALDYWGQGTTVTVSSGSFVPVFLPAKRT |
| | | TTPAPRPPTPAPTIASQPLSLRPEACRPAAGGAVHTRGLDFA |
| | | CDIYIWAPLAGTCGVLLLSLVITLYCRSKRSRLLHSDYMNM |
| | | TPRRPGPTRKHYQPYAPPRDFAAYRSRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQG |
| | | LSTATKDTYDALHMQALPPRGSGATNFSLLKQAGDVEENP |
| | | GPMRISKPHLRSISIQCYLCLLLNSHFLTEAGIHVFILGCFSA |
| | | GLPKTEANWVNVISDLKKIEDLIQSMHIDATLYTESDVHPS |
| | | CKVTAMKCFLLELQVISLESGDASIHDTVENLIILANNSLSS |
| | | NGNVTESGCKECEELEEKNIKEFLQSFVHIVQMFINTSGSG |
| | | EGRGSLLTCGDVEENPGPMWQLLLPTALLLLVSAGMRTED |
| | | LPKAVVFLEPQWYRVLEKDSVTLKCQGAYSPEDNSTQWF |
| | | HNESLISSQASSYFIDAATVDDSGEYRCQTNLSTLSDPVQL |
| | | EVHIGWLLLQAPRWVFKEEDPIHLRCHSWKNTALHKVTY |
| | | LQNGKGRKYFHHNSDFYIPKATLKDSGSYFCRGLVGSKNV |
| | | SSETVNITITQGLAGSTISSFFPPGYQVSFCLVMVLLFAVDT |
| | | GLYFSVKTNIRSSTRDWKDHKFKWRKDPQDK |

### 7.2. ADCC Activity Assays

The ADCC activity of the 1916 cells, which express the CD16a protein with the point mutation, was verified by using the 1916 cells alone and using the 1916 cells in combination with cetuximab to kill HCT-116-Luc cells. Unedited NK cells were used as a negative control, and an NK cell alone group and an NK cell-cetuximab combination group were set. The effector-to-target ratio (E:T) was set to 10:1, 5:1, 2.5:1, and 1.25:1 for the experiment. The proportion of lysed cells in each group was determined using the method of Example 6.2. Subsequently, the increase in the proportion of lysed target cells in the 1916 cell-cetuximab combination group relative to the 1916 cell alone group and, in the negative control, the increase in the proportion of lysed target cells in the unedited NK cell-cetuximab combination group relative to the unedited NK cell alone group were calculated, and the results are shown in FIG. 8.

As CD16a is complementary to the receptor-binding region in the Fc of cetuximab, its binding stimulates the ADCC of cetuximab. FIG. 8 shows that after being co-cultured with cetuximab for 4 h at the different effector-to-target ratios, the 1916 cells significantly improved the ADCC activity of cetuximab: their proportion of killed cells was higher and significantly superior to that of the iPSC-derived unedited NK cells. It can be seen that the mutated CD16a protein exhibited an excellent ADCC-stimulating effect on the iPSC-derived NK cells.

1. A cell, wherein the cell is genetically modified to express an amino acid-modified CD16 protein.

2. The cell according to claim 1, wherein the amino acid-modified CD16 protein comprises one or more amino acid additions, deletions, or substitutions or any combination of amino acid additions, deletions, and substitutions compared to the amino acid sequence of a wild-type CD16 protein; preferably, wherein the one or more amino acid substitutions include a substitution of the glutamine residue at position 192, a substitution of the leucine residue at position 194, a substitution of the valine residue at position 196, a substitution of the threonine residue at position 198, a substitution of the isoleucine residue at position 199, and/or a substitution of the serine residue at position 200 of SEQ ID NO. 3; more preferably, wherein the one or more amino acid substitutions include Q192P, L194P, V196P, T198P, I199P, S200P, L194Y, L194V, L194K, L194I, A195V, V196E, V196D, V196K, V196N, V196G, V196R, V196Q, V196M, V196H, T191S, Q192N, Q192K, A195G, V196S, T198S, I199L, and/or S200T.

3. (canceled)

4. (canceled)

5. The cell according to claim 1, wherein the amino acid-modified CD16 protein comprises an amino acid sequence set forth in any one of SEQ ID NOs: 4-22 and 24-32 or an amino acid sequence having at least 80%, at least 85%, at least 90%, at least 95%, at least 96%, at least 97%, at least 98%, at least 99%, or 100% sequence identity to the amino acid sequence set forth in any one of SEQ ID NOs: 4-22 and 24-32.

6. The cell according to claim 1, wherein the cell further expresses a chimeric antigen receptor (CAR); preferably, the CAR specifically targets BCMA and GPRC5D, more preferably, wherein the CAR targeting BCMA and GPRC5D comprises an amino acid sequence set forth in SEQ ID NO: 46.

7. (canceled)

8. The cell according to claim 6, wherein the cell further expresses an IL-15 protein, and the IL-15 protein comprises an amino acid sequence set forth in SEQ ID NO: 47; optionally, wherein the cell expresses a fusion polypeptide comprising the CAR targeting BCMA and GPRC5D and the CD16 protein; the fusion polypeptide comprises an amino acid sequence set forth in any one of SEQ ID NOs: 51-62 and 64-66 or an amino acid sequence having at least 80%, at least 85%, at least 90%, at least 95%, at least 96%, at least 97%, at least 98%, at least 99%, or 100% sequence identity to the amino acid sequence set forth in any one of SEQ ID NOs. 51-62 and 64-66.

9. (canceled)

10. The cell according to claim 1, wherein the cell is a T cell, a natural killer (NK) cell, a peripheral blood mononuclear cell (PBMC), a hematopoietic cell, a multipotent stem cell, or an embryonic stem cell; preferably, the cell is an NK cell; more preferably, the cell is an NK92 cell; optionally, wherein the amino acid-modified CD16 protein has an anti-cleavage ability.

11. (canceled)

12. A therapeutic composition, comprising the cell according to claim 1 or a cell population comprising the cell.

13. The therapeutic composition according to claim 12, wherein the therapeutic composition further comprises an iPSC cell population, an NK cell population, or a T cell population; preferably, the iPSC cell population, the NK cell population, or the T cell population is genetically modified to (i) specifically recognize a tumor antigen or (ii) specifically recognize a viral target; optionally, wherein the therapeutic composition further comprises an additional therapeutic agent; preferably, the additional therapeutic agent is an antineoplastic agent; more preferably, the antineoplastic agent is a monoclonal antibody; more preferably, the monoclonal antibody is daratumumab or cetuximab.

14. (canceled)

15. A method for treating a patient in need thereof, comprising administering to the patient the cell according to claim 1, or the therapeutic composition comprising the cell according to claim 1 or a cell population comprising the cell.

16. (canceled)

17. The method according to claim 15, wherein the method is used for inhibiting tumor cell proliferation; preferably, the tumor cell is a solid tumor cell or a hematologic tumor cell; optionally, the method further comprises administering to the patient an additional therapeutic agent; preferably, the agent is selected from a monoclonal antibody, a polyclonal antibody, a small-molecule therapeutic agent, an antibody-drug conjugate, and a cytokine; more preferably, the agent inhibits tumor cell proliferation.

18. (canceled)

19. A medicament or kit, comprising the cell according to claim 1, or the therapeutic composition comprising the cell according to claim 1 or a cell population comprising the cell.

20. An anti-cleavage recombinant CD16 protein, comprising one or more amino acid additions, deletions, or substitutions or any combination of amino acid additions, deletions, and substitutions compared to a wild-type amino acid sequence of a CD16 protein; preferably wherein the CD16 protein is derived from a human a rat, a mouse, a monkey, a pig or a dog; and preferably, the amino acid sequence of the wild-type CD16 protein is selected from SEQ ID NOS: 1 and 3.

21. (canceled)

22. The protein according to claim 20, wherein the one or more amino acid substitutions include a substitution of the glutamine residue at position 192, a substitution of the leucine residue at position 194, a substitution of the valine residue at position 196, a substitution of the threonine residue at position 198, a substitution of the isoleucine residue at position 199, and/or a substitution of the serine residue at position 200 of SEQ ID NO: 3; preferably, wherein the one or more amino acid substitutions include Q192P, L194P, V196P, T198P, I199P, S200P L194Y, L194V, L194K, L194I, A19SV, V196E, V196D, V196K, V196N, V196G, V196R V196Q, V196M, V196H, T191S, Q192N, Q192K, A195G, V196S, T198S, I199L, and/or S200T.

23. (canceled)

24. The protein according to claim 20, wherein the protein comprises an amino acid sequence set forth in any one of SEQ ID NOs: 4-22 and 24-32 or an amino acid sequence having at least 80%, at least 85%, at least 90%, at least 95%, at least 96%, at least 97%, at least 98%, at least 99%, or 100% sequence identity to the amino acid sequence set forth in any one of SEQ ID NOs: 4-22 and 24-32.

25. A fusion polypeptide, comprising the recombinant CD16 protein according to claim 20.

26. The fusion polypeptide according to claim 25, wherein the fusion polypeptide further comprises a CAR sequence targeting BCMA and GPRC5D; preferably, the CAR sequence targeting BCMA and GPRC5D comprises an amino acid sequence set forth in SEQ ID NO: 46; optionally, wherein the fusion polypeptide further comprises an IL-15 protein sequence preferably, the IL-15 protein sequence comprises an amino acid sequence set forth in SEQ ID NO: 47.

27. (canceled)

28. The fusion polypeptide according to claim 25, wherein the fusion polypeptide comprises an amino acid sequence set forth in any one of SEQ ID NOs: 51-62 and 64-66 or an amino acid sequence having at least 80%, at least 85%, at least 90%, at least 95%, at least 96%, at least 97%, at least 98%, at least 99%, or 100% sequence identity to the amino acid sequence set forth in any one of SEQ ID NOs: 51-62 and 64-66

29. A nucleic acid molecule, wherein the nucleic acid molecule encodes the protein according to claim 20, or the fusion polypeptide comprising the recombinant CD16 protein according to claim 20.

30. An expression vector, comprising the nucleic acid molecule according to claim 29.

31. A host cell, comprising the vector according to claim 30, wherein preferably, the cell is a prokaryotic cell or a eukaryotic cell, such as a bacterium (E. coli), a fungus (yeast), an insect cell, or a mammalian cell (a CHO cell line or a 293T cell line).


SEQUENCE LISTING (72 sequences)

SEQ ID NO: 1 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 2 - DNA, 765 bp, synthetic construct
  source 1..765: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 3 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 4 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITPGLAVSTISSFFPPGYQVSFCLVMVLLFAVDTGLYFSVKTNIRSSTRDWKDHKFKWRKDPQDK

SEQ ID NO: 5 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPAVSTISSFFPPGYQVSFCLVMVLLFAVDTGLYFSVKTNIRSSTRDWKDHKFKWRKDPQDK

SEQ ID NO: 6 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAPSTISSFFPPGYQVSFCLVMVLLFAVDTGLYFSVKTNIRSSTRDWKDHKFKWRKDPQDK

SEQ ID NO: 7 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSPISSFFPPGYQVSFCLVMVLLFAVDTGLYFSVKTNIRSSTRDWKDHKFKWRKDPQDK

SEQ ID NO: 8 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTPSSFFPPGYQVSFCLVMVLLFAVDTGLYFSVKTNIRSSTRDWKDHKFKWRKDPQDK

SEQ ID NO: 9 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTIPSFFPPGYQVSFCLVMVLLFAVDTGLYFSVKTNIRSSTRDWKDHKFKWRKDPQDK

SEQ ID NO: 10 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGYAVSTISSFFPPGYQVSFCLVMVLLFAVDTGLYFSVKTNIRSSTRDWKDHKFKWRKDPQDK

SEQ ID NO: 11 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGVAVSTISSFFPPGYQVSFCLVMVLLFAVDTGLYFSVKTNIRSSTRDWKDHKFKWRKDPQDK

SEQ ID NO: 12 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGKAVSTISSFFPPGYQVSFCLVMVLLFAVDTGLYFSVKTNIRSSTRDWKDHKFKWRKDPQDK

SEQ ID NO: 13 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLVVSTISSFFPPGYQVSFCLVMVLLFAVDTGLYFSVKTNIRSSTRDWKDHKFKWRKDPQDK

SEQ ID NO: 14 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAESTISSFFPPGYQVSFCLVMVLLFAVDTGLYFSVKTNIRSSTRDWKDHKFKWRKDPQDK

SEQ ID NO: 15 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLADSTISSFFPPGYQVSFCLVMVLLFAVDTGLYFSVKTNIRSSTRDWKDHKFKWRKDPQDK

SEQ ID NO: 16 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAKSTISSFFPPGYQVSFCLVMVLLFAVDTGLYFSVKTNIRSSTRDWKDHKFKWRKDPQDK

SEQ ID NO: 17 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLANSTISSFFPPGYQVSFCLVMVLLFAVDTGLYFSVKTNIRSSTRDWKDHKFKWRKDPQDK

SEQ ID NO: 18 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAGSTISSFFPPGYQVSFCLVMVLLFAVDTGLYFSVKTNIRSSTRDWKDHKFKWRKDPQDK

SEQ ID NO: 19 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLARSTISSFFPPGYQVSFCLVMVLLFAVDTGLYFSVKTNIRSSTRDWKDHKFKWRKDPQDK

SEQ ID NO: 20 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAQSTISSFFPPGYQVSFCLVMVLLFAVDTGLYFSVKTNIRSSTRDWKDHKFKWRKDPQDK

SEQ ID NO: 21 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAMSTISSFFPPGYQVSFCLVMVLLFAVDTGLYFSVKTNIRSSTRDWKDHKFKWRKDPQDK

SEQ ID NO: 22 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAHSTISSFFPPGYQVSFCLVMVLLFAVDTGLYFSVKTNIRSSTRDWKDHKFKWRKDPQDK

SEQ ID NO: 23 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDK

SEQ ID NO: 24 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITISQGLAVSTISSFFPPGYQVSFCLVMVLLFAVDTGLYFSVKTNIRSSTRDWKDHKFKWRKDPQDK

SEQ ID NO: 25 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITNGLAVSTISSFFPPGYQVSFCLVMVLLFAVDTGLYFSVKTNIRSSTRDWKDHKFKWRKDPQDK

SEQ ID NO: 26 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITKGLAVSTISSFFPPGYQVSFCLVMVLLFAVDTGLYFSVKTNIRSSTRDWKDHKFKWRKDPQDK

SEQ ID NO: 27 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGIAVSTISSFFPPGYQVSFCLVMVLLFAVDTGLYFSVKTNIRSSTRDWKDHKFKWRKDPQDK

SEQ ID NO: 28 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLGVSTISSFFPPGYQVSFCLVMVLLFAVDTGLYFSVKTNIRSSTRDWKDHKFKWRKDPQDK

SEQ ID NO: 29 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLASSTISSFFPPGYQVSFCLVMVLLFAVDTGLYFSVKTNIRSSTRDWKDHKFKWRKDPQDK

SEQ ID NO: 30 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSSISSFFPPGYQVSFCLVMVLLFAVDTGLYFSVKTNIRSSTRDWKDHKFKWRKDPQDK

SEQ ID NO: 31 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTLSSFFPPGYQVSFCLVMVLLFAVDTGLYFSVKTNIRSSTRDWKDHKFKWRKDPQDK

SEQ ID NO: 32 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTITSFFPPGYQVSFCLVMVLLFAVDTGLYFSVKTNIRSSTRDWKDHKFKWRKDPQDK

SEQ ID NO: 33 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQHHHHHH

SEQ ID NO: 34 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQHHHHHH

SEQ ID NO: 35 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITPGLAVSTISSFFPPGYQHHHHHH

SEQ ID NO: 36 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPAVSTISSFFPPGYQHHHHHH

SEQ ID NO: 37 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLVVSTISSFFPPGYQHHHHHH

SEQ ID NO: 38 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAGSTISSFFPPGYQHHHHHH

SEQ ID NO: 39 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAQSTISSFFPPGYQHHHHHH

SEQ ID NO: 40 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAMSTISSFFPPGYQHHHHHH

SEQ ID NO: 41 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAHSTISSFFPPGYQHHHHHH

SEQ ID NO: 42 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQHHHHHH

SEQ ID NO: 43 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITNGLAVSTISSFFPPGYQHHHHHH

SEQ ID NO: 44 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGIAVSTISSFFPPGYQHHHHHH

SEQ ID NO: 45 - protein, 214 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLASSTISSFFPPGYQHHHHHH

SEQ ID NO: 46 - protein, 714 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 48 - protein, 19 aa, synthetic construct
ATNFSLLKQAGDVEENPGP

SEQ ID NO: 49 - protein, 18 aa, synthetic construct
EGRGSLLTCGDVEENPGP

SEQ ID NO: 50 - protein, 898 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 51 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 52 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITPGLAVSTISSFFPPGYQVSFCLVMVLLFAVDTGLYFSVKTNIRSSTRDWKDHKFKWRKDPQDK

SEQ ID NO: 53 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPAVSTISSFFPPGYQVSFCLVMVLLFAVDTGLYFSVKTNIRSSTRDWKDHKFKWRKDPQDK

SEQ ID NO: 54 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSPISSFFPPGYQVSFCLVMVLLFAVDTGLYFSVKTNIRSSTRDWKDHKFKWRKDPQDK

SEQ ID NO: 55 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGYAVSTISSFFPPGYQVSFCLVMVLLFAVDTGLYFSVKTNIRSSTRDWKDHKFKWRKDPQDK

SEQ ID NO: 56 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGVAVSTISSFFPPGYQVSFCLVMVLLFAVDTGLYFSVKTNIRSSTRDWKDHKFKWRKDPQDK

SEQ ID NO: 57 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGKAVSTISSFFPPGYQVSFCLVMVLLFAVDTGLYFSVKTNIRSSTRDWKDHKFKWRKDPQDK

SEQ ID NO: 58 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLVVSTISSFFPPGYQVSFCLVMVLLFAVDTGLYFSVKTNIRSSTRDWKDHKFKWRKDPQDK

SEQ ID NO: 59 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAGSTISSFFPPGYQVSFCLVMVLLFAVDTGLYFSVKTNIRSSTRDWKDHKFKWRKDPQDK

SEQ ID NO: 60 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAQSTISSFFPPGYQVSFCLVMVLLFAVDTGLYFSVKTNIRSSTRDWKDHKFKWRKDPQDK

SEQ ID NO: 61 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAMSTISSFFPPGYQVSFCLVMVLLFAVDTGLYFSVKTNIRSSTRDWKDHKFKWRKDPQDK

SEQ ID NO: 62 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAHSTISSFFPPGYQVSFCLVMVLLFAVDTGLYFSVKTNIRSSTRDWKDHKFKWRKDPQDK

SEQ ID NO: 63 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDK

SEQ ID NO: 64 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITNGLAVSTISSFFPPGYQVSFCLVMVLLFAVDTGLYFSVKTNIRSSTRDWKDHKFKWRKDPQDK

SEQ ID NO: 65 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGIAVSTISSFFPPGYQVSFCLVMVLLFAVDTGLYFSVKTNIRSSTRDWKDHKFKWRKDPQDK

SEQ ID NO: 66 - protein, 1176 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSLEGGGEGRGSLLTCGDVEENPGPRMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLASSTISSFFPPGYQVSFCLVMVLLFAVDTGLYFSVKTNIRSSTRDWKDHKFKWRKDPQDK

SEQ ID NO: 67 - protein, 504 aa, synthetic construct
MALPVTALLLPLALLLHAARPLEEIVLTQSPATLSLSPGERATLSCSASSSISSNYLHWYQQKPGQAPRFLIYRTSNLASGIPARFSGSGSGTDFTLTISSLEPEDFAVYYCQQASSIPRMFTFGQGTKLEIKGSTSGSGKPGSGEGSTKGEVQLQQSGAEVKKPGASVKVSCKASGYTFTNYWMHWVRQRPGQGLEWMGEIDPSDNYANYNQEFQGRVTITVDKSASTAYMELSSLRSEDTAVYYCARHDGYFGALDYWGQGTTVTVSSGSFVPVFLPAKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 162 aa, synthetic construct; identical to SEQ ID NO: 47

SEQ ID NO: 69 - protein, 19 aa, synthetic construct
ATNFSLLKQAGDVEENPGP

SEQ ID NO: 70 - protein, 18 aa, synthetic construct
EGRGSLLTCGDVEENPGP

SEQ ID NO: 71 - protein, 688 aa, synthetic construct
MALPVTALLLPLALLLHAARPLEEIVLTQSPATLSLSPGERATLSCSASSSISSNYLHWYQQKPGQAPRFLIYRTSNLASGIPARFSGSGSGTDFTLTISSLEPEDFAVYYCQQASSIPRMFTFGQGTKLEIKGSTSGSGKPGSGEGSTKGEVQLQQSGAEVKKPGASVKVSCKASGYTFTNYWMHWVRQRPGQGLEWMGEIDPSDNYANYNQEFQGRVTITVDKSASTAYMELSSLRSEDTAVYYCARHDGYFGALDYWGQGTTVTVSSGSFVPVFLPAKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 72 - protein, 963 aa, synthetic construct
MALPVTALLLPLALLLHAARPLEEIVLTQSPATLSLSPGERATLSCSASSSISSNYLHWYQQKPGQAPRFLIYRTSNLASGIPARFSGSGSGTDFTLTISSLEPEDFAVYYCQQASSIPRMFTFGQGTKLEIKGSTSGSGKPGSGEGSTKGEVQLQQSGAEVKKPGASVKVSCKASGYTFTNYWMHWVRQRPGQGLEWMGEIDPSDNYANYNQEFQGRVTITVDKSASTAYMELSSLRSEDTAVYYCARHDGYFGALDYWGQGTTVTVSSGSFVPVFLPAKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGEGRGSLLTCGDVEENPGPMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAGSTISSFFPPGYQVSFCLVMVLLFAVDTGLYFSVKTNIRSSTRDWKDHKFKWRKDPQDK